(19) United States
(12) Patent Application Publication
(10) Pub. No.: US 2009/0214660 A1
(43) Pub. Date: Aug. 27, 2009

(54) ENCAPSULATION SYSTEM

(75) Inventors: Alfred Vasconcellos, Cranston, RI (US); Dwaine Emerich, N. Scituate, RI (US); Chris Thanos, Cumberland, RI (US); Briannan Bintz, Cumberland, RI (US); Marilyn Sandra Geaney, Auckland (NZ); Stephen John Martin Skinner, Auckland (NZ); Paul Lip Jin Tan, Auckland (NZ)

Correspondence Address: GREENLEE WINNER AND SULLIVAN P C, 4875 PEARL EAST CIRCLE, SUITE 200, BOULDER, CO, 80301, US

(73) Assignee: Living Cell Products Pty Limited, Sydney, NSW (AU)

(21) Appl. No.: 12/090,981

(22) PCT Filed: Oct. 24, 2006
(86) PCT No.: PCT/NZ2006/000270

§ 371 Date: Jan. 12, 2009

Related U.S. Application Data
(60) Provisional application No. 60/729,422, filed on Oct. 21, 2005.

(51) Int. Cl. A61K 9/50; C08B 37/04; A61K 35/12

(52) U.S. Cl. 424/490; 536/123.1; 424/93.7

(57) ABSTRACT

The present invention is directed to a composition comprising high mannuronic acid-containing alginate and a polycation having a polydispersity index of less than 1.5. The composition is particularly useful for making biocompatible microcapsules containing living cells for allo- or xeno-transplantation. Such microcapsules have enhanced durability and can maintain their structural and functional integrity over long periods of time compared to prior art alginate microcapsules.

### FIELD OF THE INVENTION

The invention relates to an encapsulation system comprising alginate biocapsules for the immunoisolation of living cells or therapeutics. Specifically, although by no means exclusively, the encapsulation system is for use in allo- and xeno-transplantation. The invention is also directed to methods of making and using the encapsulation system.

### BACKGROUND OF THE INVENTION

Cell transplantation is becoming increasingly more successful both experimentally and clinically. One iteration of cell transplantation takes advantage of developments in material science, cell biology, and drug delivery to develop micro- and macro-encapsulated cell therapy platforms. These include 2-D and 3-D tissue engineered conformations composed of nonerodible thermoplastic polymers, bioerodible materials, and hybrid combinations. These constructs allow for the controlled delivery of therapeutic molecules for the treatment of acute and chronic diseases, but their widespread use is precluded by the need for frequent administration for erodible materials, and retrieval and chronic biocompatibility issues for nondegradable materials. In the case of biodegradable materials, the success of encapsulated cell therapy will depend to a large degree on an understanding of the stability of the material once transplanted and ultimately how that stability impacts the ability of the graft to support cell survival, protein secretion and diffusion, immunoisolation, biocompatibility, physical placement and fixation, degradation, and the efficacy and pharmacodynamics of the secreted product. One of the most common materials used for such biocapsules for cell therapy is alginate, a bioerodible carbohydrate.

Alginate has long been studied as a biomaterial in a wide range of physiologic and therapeutic applications. Its potential as a biocompatible implant material was first explored in 1964 in the surgical role of artificially expanding plasma volume (1). More than a decade later, the matrix capability of alginate for cell support was realized in vitro in a series of experiments that demonstrated microbial cell survival for 23 days (2). Over the last twenty years, there has been remarkable progress in alginate cell microencapsulation for the treatment of diabetes (3-10), chronic pain (11), hemophilia (12; 13), central nervous system (CNS) disorders (14-24), and others. Despite success in numerous animal models and in limited clinical allotransplantation, there have been variable degradation kinetics impacting diffusion, immunoisolation, and ultimately leading to loss of graft survival and rejection. Some well designed studies have been carried out to characterize and control certain aspects of alginate degradation in vitro (25-30) and in vivo (31; 32), but the general understanding of the stability of alginate-polycation capsules in vivo from a strict materials perspective is limited and this in turn limits their use.

It is an object of the present invention to go some way towards furthering the understanding of the stability of alginate-polycation biocapsules to produce more stable biocapsules for in vivo applications and/or to provide the public with a useful choice.

### SUMMARY OF THE INVENTION

The invention is directed to a biodurable composition comprising alginate which has a high mannuronic acid content, and a polycation which has a polydispersity index of <1.5 for producing microcapsules. Such microcapsules may be produced by standard methods. The composition of the present invention is advantageous over known compositions as it can be used to produce microcapsules that are more durable than known microcapsules and thus may allow for prolonged protection from the host immune system when discordant cells are encapsulated. This is demonstrated herein, whereby a decreased rate of degradation in vivo was observed for microcapsules composed of the composition of the present invention. The microcapsules also exhibit enhanced surface morphology and may be administered to sites which, previously, were hyperinflammatory, as set out below.

In a first aspect, the invention provides a composition comprising alginate containing between from about 50% to about 95%, preferably from about 50% to about 90%, more preferably from about 50% to about 70%, and most preferably from about 60% to about 70% mannuronic acid residues and a polycation such as poly-L-ornithine. In a preferred embodiment, the high mannuronic acid alginate and the polycation are in a ratio of approximately 5:1 to about 10:1, preferably around 7:1. In addition, the composition of the present invention may include calcium chloride and sodium chloride. In one embodiment, the composition may comprise a high mannuronic acid alginate at a concentration of about 80% to about 90%, and preferably from about 85% to about 90% and more preferably, about 87%; poly-L-ornithine at a concentration of about 10% to about 15%, preferably about 13%; calcium chloride at a concentration of less than about 1%; and sodium chloride at a concentration of less than about 1%.

The polycation, for example poly-L-ornithine, is present in the composition in a relatively purified form whereby the range of molecular weight species is limited and the polydispersity index (i.e. average MW÷median MW) is less than 1.5, preferably less than 1.2, most preferably less than 1.1.

In a second aspect, the invention provides biocompatible microcapsules prepared using the composition of the invention, and comprising a core layer of high mannuronic acid alginate cross-linked with a cross-linking agent, such as calcium ions, an intermediate layer of polycations having a polydispersity index of <1.5 forming a semi-permeable membrane, and an outer layer of high mannuronic acid alginate. The core layer and the outer layer may comprise the same or different high mannuronic acid alginate.

The microcapsules may further comprise living cells within the core layer. The cells may comprise naturally occurring or genetically engineered cells which may be in the form of single cells or cell clusters selected from the group comprising β islet cells, hepatocytes, neuronal cells such as choroid plexus cells, pituitary cells, chromaffin cells, chondrocytes, and any other cell type capable of secreting factors that would be useful in the treatment of a disease or condition.

In a third aspect, the present invention comprises a method for preparing biocompatible microcapsules comprising the steps:
a) dissolving a high mannuronic acid containing alginate in isotonic saline;
b) spraying the dissolved alginate solution of step a) through an air- or frequency-based droplet generator into a stirring solution of an excess of a cross-linking agent, such as for example, about 15 to about 120 mM, and more preferably from about 40 to about 110 mM, and more preferably still from about 90 to 110 mM calcium chloride, for about 5 to 30 minutes, preferably for 5 to 10 minutes to form gelled capsules;
c) coating the gelled capsules of step b) with a polycation having a polydispersity index of <1.5, such as poly-L-ornithine at a concentration of between about 0.02 to about 0.01% (w/v), preferably 0.05% (w/v), for between about 5 to 30 minutes, (preferably for about 10 minutes);
d) applying a final high mannuronic acid alginate coating to the capsule of step c) for between 5 and 30 minutes, (preferably for between about 5 and 10 minutes); and
e) collecting the microcapsules;
wherein the alginate used in steps a) and d) is the same or different and contains between about 50% to about 95% mannuronic acid residues, preferably between about 50% to about 90%, more preferably between about 50% to about 70%, and most preferably between about 60% and 70% mannuronic acid residues.

The alginate solution of step a) comprises an alginate concentration of about 1.0% to 2.0% w/v.

The alginate solution of step d) comprises an alginate concentration of about 0.01 to 1.7% w/v.

In a fourth aspect, the present invention comprises a method of preparing microencapsulated cells comprising the steps:
a) incubating living cells with a solution of high mannuronic acid containing alginate dissolved in isotonic saline;
b) spraying the cell-alginate solution of step a) through an air- or frequency-based droplet generator into a stirring solution of an excess of a cross-linking agent, such as about 15 mM to about 120 mM calcium chloride (preferably 110 mM), for about 5 to about 30 minutes (preferably 5-10 minutes) to form gelled cell-containing capsules;
c) coating the gelled cell-containing capsules of step b) with a polycation having a polydispersity index of <1.5, such as poly-L-ornithine, at a concentration of between about 0.02% to 0.1% (w/v) (preferably 0.05% w/v) for between about 5 and 30 minutes (preferably for about 10 minutes);
d) applying a final alginate coating to the cell-containing capsules of step c) for between about 5 and 30 minutes (preferably about 10 minutes); and
e) collecting the cell-containing microcapsules;
wherein the alginate used in steps a) and d) is the same or different and contains between about 50% to about 95% mannuronic acid residues, preferably between about 50% to about 90%, more preferably between about 50% to about 70%, and most preferably between about 60% and 70% mannuronic acid residues.

The alginate solution of step a) comprises an alginate concentration of about 1.0% to 2.0% w/v.

The alginate solution of step d) comprises an alginate concentration of about 0.01 to 1.7% w/v.

In a fifth aspect, the invention provides a method for coating non-degradable cell delivery constructs comprising the steps a) immersing the non-degradable cell delivery constructs in a solution of alginate containing between about 50 and about 95% mannuronic acid residues (preferably between about 50 and 90%, more preferably between about 50 and 70% and most preferably about 60% and 70% mannuronic acid) and isotonic saline; b) crosslinking the mannuronic acid residues by incubating in an excess of a cross-linking agent, such as a 15 mM to 120 nm solution of calcium chloride (preferably 110 mM), for about 5 to about 30 minutes (preferably between about 5 and 10 minutes) to form a gelled coating; c) further coating the gelled constructs of step b) with a polycation having a polydispersity index of less than 1.5, for example poly-L-ornithine, at a concentration of between about 0.02 and 0.1% w/v, (preferably 0.05% w/v), for between about 5 and 30 minutes, (preferably about 10 minutes); d) applying a final alginate coating for between about 5 to 30 minutes, (preferably about 10 minutes), to produce immunoisolatory membrane coated non-degradable cell delivery constructs; and e) isolating the final immunoisolatory membrane coated non-degradable cell delivery constructs.

The alginate solution of step a) comprises an alginate concentration of about 1.0% to 2.0% w/v.

The alginate solution of step d) comprises an alginate concentration of about 0.01 to 1.7% w/v.

In a sixth aspect, the invention provides a method for encapsulating small molecule, protein or DNA therapeutics comprising the steps a) dispersing the therapeutics in a solution of alginate containing a high proportion of mannuronic acid residues dissolved in isotonic saline; b) crosslinking the mannuronic acid residues by incubation in an excess of a cross-linking agent, such as a 15 mM-120 mM solution of calcium chloride (preferably 10 mM), for about 5 to 30 minutes (preferably about 10 minutes) to form gelled therapeutic-containing capsules; c) coating the gelled therapeutic-containing capsules with a polycation having a polydispersity index of less than 1.5, for example poly-L-ornithine, at a concentration of about 0.02 to 0.1% w/v, (preferably 0.05% w/v) for about 5 to 30 minutes, (preferably 10 minutes); d) applying a final alginate coating to the therapeutic-containing capsules of step c) for between 5 to 30 minutes, (preferably about 10 minutes), and e) collecting the therapeutic-containing microcapsules.

The alginate used in steps a) and d) is the same or different and contains between about 50% to about 95% mannuronic acid residues, preferably between about 50% to about 90%, more preferably between about 50% to about 70%, and most preferably between about 60% and 70% mannuronic acid residues.

The alginate solution of step a) comprises an alginate concentration of about 1.0% to 2.0% w/v.

The alginate solution of step d) comprises an alginate concentration of about 0.01 to 1.7% W/V.

In a seventh aspect, the invention provides a method of ameliorating or treating a disease or condition in an animal, including a human, comprising transplanting an effective amount of the cell-containing microcapsules of the invention into said animal, wherein said cells secrete a therapeutic that is effective at ameliorating or treating said disease or condition.

In an eighth aspect, the invention provides a method of ameliorating or treating a disease or condition in an animal, including a human, comprising transplanting an effective amount of the therapeutic-containing microcapsules of the invention into said animal, wherein said therapeutic is effective at ameliorating or treating said disease or condition.

In an ninth aspect, the invention provides a use of an alginate containing between about 50 and about 95% mannuronic acid residues and a polycation in the manufacture of a microcapsule preparation for use in allo- or xeno-transplantation applications.

The microcapsule preparations of the invention may be administered to a subject. A “subject” as used herein shall mean a human or vertebrate mammal including but not limited to a dog, cat, horse, cow, pig, sheep, goat, or primate, e.g., monkey. The microcapsule preparations comprise cells that secrete therapeutic agents or contain therapeutic agents per se and are administered in an amount sufficient to provide an effective amount of the therapeutic agent to the subject. An effective amount of a particular agent will depend on factors such as the type of agent, the purpose for administration, the severity of disease if a disease is being treated etc. Those of skill in the art will be able to determine effective amounts.

The term “comprising” as used in this specification and claims means “consisting at least in part of”, that is to say when interpreting independent claims including that term, the features prefaced by that term in each claim all need to be present but other features can also be present.

### DESCRIPTION OF THE DRAWINGS

The invention will now be described with reference to the figures of the accompanying drawings in which:

FIG. 1 shows a protein NMR spectrum of alginate at 90° C., wherein peaks are shifted downfield due to temperature and the chemical structure of alginate (see boxed insert) with the location of the protons responsible for the NMR peaks;

FIG. 2a shows FTIR of material components prior to encapsulation and the adsorptions of the carbonyl region in high magnification (see boxed insert);

FIG. 2b shows alginate mixtures with varying poly-L-ornithine (PLO) concentrations whereby the highlighted region represents the PLO amide II absorption;

FIG. 2c shows a quantitative FTIR measuring the ratio of PLO amide absorption to alginate co-absorption;

FIG. 3 shows 5× magnification phase-contrast image of VPMG capsules prior to implantation;

FIG. 4 shows 5× magnification-phase-contrast micrographs for 60-day explant specimens for different alginate types;

FIG. 5 shows the cross-sectional uniformity (A) and the % original diameter (B) for the 60-day explant specimens for FIG. 4, mean ISD. (Δ) VPMG; (⋄) VPLG; (-) pKel; (m)ₚFlu; ()pMan;

FIG. 6 shows FTIR, 1590 cm⁻¹ and 1550 cm⁻¹ peaks for each capsule group over the 90-day study period;

FIG. 7 shows quantitative FTIR stability index as a measure of the alginate carboxylic acid peak to the onithine amide II peak (Δ) VPMG; (⋄) VPLG; (-) pKel; (□)pFlu; ()pMan;

FIG. 8 shows photomicrographs of lyophilized alginate capsule surfaces for each of the alginate types VPMG, VPLG, pKel, pFlu and pMan over the 90 day study period; and

FIG. 9 shows a higher magnification of a photomicrograph to show the surface pitting of a pKel microcapsule at day 30.

### DETAILED DESCRIPTION OF THE INVENTION

The present invention is directed to an encapsulation system for living cells and therapeutics which has improved biostability when the encapsulated cells and therapeutics are implanted into a subject. This improved biostability enables the encapsulated cells and therapeutics to remain within a living body for longer periods than is currently the case which will result in improved therapeutic delivery and thus treatment efficacy.

The encapsulation system comprises a biodurable composition comprising alginate which is high in mannuronic acid.

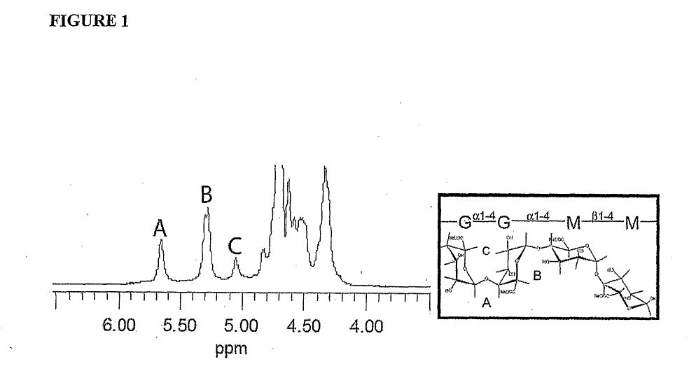

Alginate is a polysaccharide composed of guluronic (G) and mannuronic (M) acid linked by (1,4)-α- and -β-glycoside bonds (see the boxed insert in FIG. 1). The ratio of these monomers contributes directly to certain physical characteristics of the polysaccharide. It has been found for the first time that once cationically crosslinked, alginates high in G, due to a more networked structure resulting from α(1-4) bonds, are more brittle with a higher elastic modulus, while those that are high in M, with more linear β(1-4) linkages, exhibit decreased 3-D crosslinking and greater elasticity and form very stable microcapsules when tested in vivo.

Thus, the present invention provides a composition comprising a high mannuronic acid alginate, specifically containing between about 50% to 95% mannuronic acid residues, and a polycation having a polydispersity index of <1.5, such as poly-L-ornithine. Preferably the high mannuronic acid containing alginate contains between about 50% and 90% mannuronic acid residues, more preferably between about 50% and 70% mannuronic acid residues, and most preferably between about 60% and 70% mannuronic acid residues. In a preferred embodiment, the high mannuronic acid alginate and the polycation are in a ratio of approximately 5:1 to 10:1 by weight, preferably about 7:1 by weight. In addition, the composition of the present invention may include calcium chloride and sodium chloride. Preferably, the composition comprises high mannuronic acid alginate at a concentration of about 80% to about 90%, preferably about 87%, poly-L-ornithine at a concentration of about 10% to about 15%, preferably about 13%, calcium chloride at a concentration of less than about 1% and sodium chloride at a concentration of less than about 1%.

The average molecular weight of the alginate is greater than about 400 KDa, preferably greater than about 600 KDa.

The high mannuronic acid containing alginate used in the proportions in the present invention may comprise a glucoronic acid content of between about 10 and about 40%. Thus, the ratio of M:G in the alginate useful in the present invention is from between about 1.55:1 to 9.5:1.

The alginate source is purified and contains less than 1 endotoxin unit/ml of 1.7% (w/v) alginate. Examples of commercially available alginates suitable for use in the present invention include Keltone LVCR and Pronova SLM20. However, any other alginate with suitable high mannuronic acid content (or suitable M:G ratios) can be used as a raw material for use in the present invention.

The alginate may have a pH of 7.0±0.4 when dissolved in 1.7% (w/v) saline.

The molecular weight of the polycation is also important in the structural and functional composition of the microcapsules of the invention. It has been found for the first time that a polycation having a polydispersity index of less than about 1.5, preferably less than about 1.2 and more preferably less than 1.1, together with the high mannuronic acid aliginate, results in superior microcapsules which are highly stable and can remain in vivo for long periods of time, and certainly for more than one month.

Polycatonic agents comprising a high polydispersity index and therefore including a wide range of MW species are shown to result in inferior microcapsules. This is thought to be caused by the larger MW molecules being unable to diffuse into the alginate coat resulting in a weak coating. The smaller MW molecules, on the other hand, can diffuse too rapidly into the alginate coating and can penetrate into the core and displace cells or beads within the core. A polycation with a limited range of MW species has been shown to result in superior microcapsules.

For example, when the polycation is poly-L-ornithine, or poly-L-lysine, the preferred average MW for the polycation is from between 10 to 40 KDa, more preferably between 15 to 30 KDa and most preferably around 20-25 KDa.

Preferably, the poly-L-lysine or poly-L-ornithine will contain <about 20% of molecules having a MW of 10 KDa or less and more preferably <about 10% of molecules having a MW of 10 KDa or less.

The invention further provides biocompatible microcapsules prepared using the composition of the invention, and comprising a core layer of high mannuronic acid alginate cross-linked with a cationic cross-linking agent, an intermediate layer of polycations having a polydispersity index of less than about 1.5 forming a semi-permeable membrane, and an outer layer of high mannuronic acid alginate.

The high mannuronic acid alginate may comprise from about 50% to about 95% mannuronic acid residues, preferably from about 50% to about 90%, more preferably from about 50% to about 70% and most preferably from about 60% to about 70% mannuronic acid residues.

The alginate used in the core layer and the outer layer may be the same or different.

The core layer may comprise alginate composed of 50-70% mannuronic acid residues and the outer layer may comprise alginate composed of 10-40% glucoronic acid residues.

The cationic cross-linking agent may be selected from salts of the group consisting of Ag⁺, Al³⁺, Ba²⁺, Ca²⁺, Cd²⁺, Cu²⁺, Fe²⁺, Fe³⁺, H⁺, K⁺, Li⁺, M⁺, Mn²⁺, Na⁺, N⁴⁺, Ni²⁺, Pb²⁺ Sn²⁺ and Zn²⁺. Preferably the cationic cross-linking agent is calcium chloride. The cross-linking agent is preferably in excess, for example from 15 mM to 120 mM calcium chloride. More preferably 110 mM calcium chloride.

The polycationic agent may be selected from the group consisting of chitosan glutamate, chitosan glycol, modified dextran, lysozyme, poly-L-lysine, poly-L-ornithine, salmine sulfate, protamine sulfate, polyacrylimide, polyacrylimide-co-methacryloxyethyltrimethylammonium bromide, polyallylamine, polyamide, polyamine, polybrene, Polybutylacrylate-co-Methacryloxyethyl Trimethylammonium Bromide (80/20), Poly-3-chloro-2-hydroxypropylmethacryl-oxyethyl dimethylammonium Chloride, Polydiallyldimethylammonium, Polydiallyldimethylammonium Chloride, Polydiallyldimethylammonium Chloride Acrylamide, Polydiallyldimethylammonium Chloride-co-N-Isopropyl Acrylaride, Polydimethylamine-co-epichlorohydrin, Polydimethylaminoethylacrylate-co-Acrylamide, Polydimethylaminoethylmethacrylate, Polydimethylaminoethyl Methacrylate, Polyethyleneimine, Polyethyleneimine-Epichlorohydrin Modified, Polyethyleneimine, Poly-2-hydroxy-3-methacryloxypropyl Trimethylammonium Chloride, Poly-2-hydroxy-3-methacryloxyethyl, Trimethylammonium Chloride, Polyhdroxyproplymethacryloxy Ethyldi methyl Ammonium Chloride, Polyimadazoline (Quaternary), Poly-2-methacryloxyethyltrimethylammonium Bromide, Polyniethacryloxyethyltrimethylammonium Bromide/Chloride, Polymethyldiethylaminoethylmethacrylate-co-Acrylamide, Poly-1-methyl-2-vinylpyridinium Bromide, Poly-1-methyl-4-vinylpyridinium Bromide, Polymethylene-co-Guanidine Hydrochloride, Polyvinylamine, Poly-N-vinylpyrrolidone-co-Dimethylaminoelhyl-Methacrylate, and Poly-4-vinylbenzyltrimethylammonium Chloride, and Poly-4-vinylbenzyltrimethylammonium Chloride.

Preferably the polycationic agent is poly-L-ornithine at a concentration of between 0.02% and 0.1% wv.

Poly-L-ornithine is preferably purified to remove the higher and/or lower MW species to give a polydispersity index of preferably less than 1.2 and more preferably less than 1.1. Specifically, the average MW for the poly-L-ornithine polycationic agent is from between 10 to 40 KDa, more preferably between 15 and 30 KDa and most preferably around 20 to 25 KDa. Any molecular weight molecules below 10 KDa and above 40 KDa can be removed by dialysis and other known methods. Preferably, the poly-L-ornithine used in the present invention comprises less than about 20% of molecules having a MW of 10 KDa or less and more preferably less than 10% of molecules having a MW of 10 KDa or less.

The intermediate layer, which is formed of polycations around the core layer, comprise a semipermeable membrane of between about 10 and about 80 μm in thickness.

The alginate of the core layer may be solid or may be depolymerised by a chelation agent to form a hollow core. Examples of suitable chelation agents are sodium citrate and EDTA.

It is thought that chelation of the alginate (degelling) core solubilises the internal structural support of the capsule, thereby adversely affecting the durability of the microcapsule. This problem is overcome in the prior art by not carrying out the chelation step so that the core is solid (see U.S. Pat. No. 6,365,385, for example). However, the use of a high mannuronic acid containing alginate in the microcapsules of the present invention together with the use of a polycation having a polydispersity index of less than 1.5 significantly increases the durability of the microcapsules even when the core is liquidised by chelation. The microcapsules of the present invention may also have a solid core for further enhanced stability and durability.

The ratio of the core layer of alginate to the polycationic agent is about 7:1 to about 8:1 by weight.

The ratio of the outer layer of alginate to the polycationic agent is about 1.5:1 to about 1.4:1 by weight.

The formed microcapsules swell approximately 10% or greater in volume when placed in vitro in physiological conditions for about one month or more. Swelling of microcapsules is thought to be caused by surplus divalent cations causing an osmotic gradient leading to water uptake. This can be problematic and lead to the decomposition of the microcapsules. This problem can be overcome by mopping up the excess cations with anions (as for example in U.S. Pat. No. 6,592,886). However, in the present invention, the use of a high mannuronic acid containing alginate together with the use of a polycation agent having a polydispersity index of less than 1.5 results in fewer surplus cations and the microcapsule of the invention is highly stable and less likely to decompose, although as described, there is some limited swelling.

The surface of the microcapsule when formed has an ionically neutral surface.

The microcapsules may further comprise living cells within the core layer. The cells may comprise naturally occurring or genetically engineered cells which may be in the form of single cells and/or cell clusters selected from the group consisting of β islet cells, hepatocytes, neuronal cells such as choroid plexus cells, pituitary cells, chromafin cells, chondrocytes and any other cell type capable of secreting factors that would be useful in the treatment of a disease or condition.

For example, the cells may be islet cells capable of secretory insulin useful for the treatment of diabetes.

The cells may alternatively comprise hepatocyte or non-hepatocyte cells capable of secreting liver secretory factors useful in the treatment of liver diseases or disorders.

The cells may alternatively comprise neuronal cells, such as choroids plexus, pituitary cells, chromoffin cells, chondrocytes and any other cell capable of secreting neuronal factors useful in the treatment of neuronal diseases such as Parkinson's disease, Alzheimer's disease, epilepsy, Huntington's disease, stroke, motor neurone disease, amyotrophic lateral sclerosis (ALS), multiple sclerosis, aging, vascular disease, Menkes Kinky Hair Syndrome, Wilson's disease, trauma or injury to the nervous system.

The microcapsules of the present invention may be between 50 and 2000 microns in diameter. Preferably the microcapsules are between about 100 and 1000 microns in diameter, and more preferably between about 500 and 700 microns in diameter.

It is expected that the microcapsules of the present invention will be able to remain functional in vivo in a subject for a significant period of time and certainly for periods greater than one month.

The functional duration of the microcapsules may be controlled by one or more of the following methods:
by varying the polydispersity of the alginate range used in the inner and/or outer layers of the micro capsule;
by varying the total protein content of the inner and/or outer alginate layers;
by inducing calcification of the alginate layers;
by varying the range and distribution of molecular weight of the polycationic agent;
by varying the concentration of polycationic unreacted contaminant with concentrations from about 0.01% to about 0.25% (w/w);
by varying the uniformity of the polycation concentration, creating a gradient across the intermediate layer of the microcapsule;
by varying the amount of cell-surface interaction by coating the external surface with inhibitory agents such as surfactants including pluronics F127, anti-fibrotics, and other suitable agents.

The present invention further provides a method for preparing the biocompatible microcapsules of the invention comprising the steps:
a) dissolving a high mannuronic acid containing alginate in isotonic saline;
b) spraying the dissolved alginate solution of step a) through an air- or frequency-based droplet generator into a stirring solution of an excess of a cross-linking agent, for about 5-30 minutes (preferably 5 to 10 minutes) to form gelled capsules;
c) coating the gelled capsules of step b) with a polycation having a polydispersity index of less than about 1.5, such as poly-L-ornithine, at a concentration of 0.01 to 0.2% w/v, (preferably 0.05% w/v), for 5-30 minutes (preferably 10 minutes);
d) applying a final high mannuronic acid alginate coating to the capsule of step c) for between about 5-30 minutes (preferably for between 5 and 10 minutes); and
e) collecting the microcapsules;
   wherein the high mannuronic acid containing alginate used in steps a) and d) is the same or different and contains between about 50% and about 95% mannuronic acid residues, preferably between 50 and 90%, more preferably between about 50 and 70%, and most preferably about 60% and about 70% mannuronic acid residues.

The alginate solution of step a) comprises an alginate concentration of about 1.0% to 2.0% w/v.

The alginate solution of step d) comprises an alginate concentration of about 0.01 to 1.7% w/v.

The cross-linking agent may be selected from the group listed above and is preferably about 110 mM calcium chloride.

The final alginate coating preferably contains between about 10 and about 40% glucoronic acid residues.

The alginate of the core layer may be solid or may be depolymerised by a chelation agent to form a hollow core as described above.

The present invention further provides a method of preparing microencapsulated cells comprising the steps:
a) incubating living cells with a solution of high mannuronic acid containing alginate dissolved in isotonic saline;
b) spraying the cell-alginate solution of step a) through an air- or frequency-based droplet generator into a stirring solution of an excess of a cationic cross-linking agent, such as about 15 mM to 120 mM calcium chloride (preferably 110 mM), for about 5-30 minutes (preferably 5 to 10 minutes) to form gelled cell-containing capsules;
c) coating the gelled cell-containing capsules of step b) with a polycation having a polydispersity index of less than 1.5, preferably poly-L-ornithine, at a concentration of about 0.02% to 0.1% w/v, (preferably 0.05% w/v), for between 5 to about 30 minutes (preferably about 10 minutes);
d) applying a final alginate coating the cell-containing capsules of step c) for between 5 and 30 minutes (preferably about 10 minutes); and
e) collecting the cell-containing microcapsules;
   wherein the alginate used in steps a) and d) is the same or different and contains between about 50% to about 95% mannuronic acid residues, preferably between about 50% to about 90%, more preferably between about 50% to about 70%, and most preferably between about 60% and 70% mannuronic acid residues.

The alginate solution of step a) comprises an alginate concentration of about 1.0% to 2.0% w/v.

The alginate solution of step d) comprises an alginate concentration of about 0.01 to 1.7% w/v.

The cells may be naturally occurring or genetically engineered cells which may be in he form of single cells and/or cell clusters selected from the group comprising of β islet cells, hepatocytes, neuronal cells such as choroid plexus cells, pituitary cells, chromaffin cells, chondrocytes and any other cell type capable of secreting factors that would be useful in the treatment of a disease or condition.

The cells may be isolated from the same species as a recipient host, for use in allo-transplantation, or from a different species, for use in xeno-transplantation.

The cells are preferably contained within the core alginate layer but can alternatively or additionally be contained within the outer alginate layer.

The invention further provides a method for coating non-degradable cell delivery constructs comprising the steps a) immersing the non-degradable cell delivery constructs in a solution of alginate containing between about 50 to about 95% mannuronic acid residues and isotonic saline; b) crosslinking the mannuronic acid residues by incubating with an excess of a cross-linking agent, for example a solution of about 15 mM to 120 mM (preferably 110 mM) calcium chloride, for about 5-30 minutes (preferably 5 to 10 minutes) to form a gelled coating; c) further coating the gelled constructs of step b) with a polycation having a polydispersity index of less than 1.5, preferably poly-L-ornithine at a concentration of about 0.02 to 0.1% w/v, (preferably 0.05% w/v), for about 5 to 30 minutes (preferably 10 minutes); d) applying a final alginate coating for between about 5 to 30 minutes to form immunoisolatory membrane coated non-degradable cell delivery constructs; and e) isolating the final immunoisolatory membrane coated non-degradable cell delivery constructs.

The alginate solution of step a) comprises an alginate concentration of about 1.0% to 2.0% w/v.

The alginate solution of step d) comprises an alginate concentration of about 0.01 to 1.7% w/v.

The non-degradable cell delivery construct may be selected from the group consisting of hollow-fiber membrane devices, flat sheets, porous scaffolds for cell ingrowth and other novel scaffolding constructs, as would be appreciated by a skilled worker.

The non-degradable cell delivery construct may comprise living cells which may be naturally occurring or genetically engineered cells in the form of single cells and/or cell clusters selected from β islet cells, hepatocytes, neuronal cells such as choroids plexus cells, pituatary cells, chromaffin cells, chondrocytes and any other cell type capable of secreting factors that would be useful in the treatment of a disease or condition.

The invention further provides a method for encapsulating small molecule, protein or DNA therapeutics comprising the steps a) dispersing the therapeutics in a solution of a high mannuronic acid alginate dissolved in isotonic saline; b) crosslinking the mannuronic acid residues by incubation in an excess of a cross-linking agent, preferably in a solution of about 15-120 mM calcium chloride (preferably 110 mM), for about 5 to about 30 minutes to form gelled therapeutic-containing capsules; c) coating the gelled therapeutic-containing capsules with a polycation having a polydispersity index of less than 1.5, preferably poly-L-ornithine at a concentration of about 0.02 to 0.1% w/v, (preferably 0.05% w/v), for about 5 to 30 minutes; d) applying a final alginate coating to the therapeutic-containing capsules of c) for 5 and 30 minutes, and e) collecting the therapeutic-containing microcapsules.

The alginate solution of step a) comprises an alginate concentration of about 1.0% to 2.0% w/v.

The alginate solution of step d) comprises an alginate concentration of about 0.01 to 1.7% w/v.

The small molecule, protein or DNA therapeutic is preferably contained within the core alginate layer but may alternatively or additionally be contained within the outer alginate layer.

Alternatively, the small molecule, protein or DNA therapeutic may be bound to the outer alginate layer or may be contained within the (polycationic) intermediate layer.

Examples of suitable protein therapeutics include erythropoietin, insulin, CNTF, BDNF, GDNF, GH, and others, as would be appreciated by a skilled worker.

In certain aspects, it may be desirable to utilise an alginate that contains from between about 50% to about 90% mannuronic acid residues, and in certain embodiments, a range of from between about 50% to about 70% mannuronic acid residues, and preferably between 60% to 70% mannuronic acid residues. Likewise, it may be desirable in certain aspects of the invention to apply the final alginate coating in a concentration of from about 0.05% to about 0.20% w/v. As mentioned above, the times for spraying, coating and then applying the alginate coating, may be substantially shorter or longer than about 10 minutes, and may in certain cases require about 1 to about 45 minutes for each step, while in some applications of the invention, each of these steps may be performed for a period of from about 5 to about 20 minutes each.

The invention further provides a method of ameliorating or treating a disease or condition in an animal, including a human, comprising transplanting an effective amount of the cell-containing microcapsules of the invention into said animal, wherein said cells secrete a therapeutic that is effective at ameliorating or treating said disease or condition.

The invention further provides a method of ameliorating or treating a disease or condition in an animal, including a human, comprising transplanting an effective amount of the cell-containing immunoisolatory membrane coated non-degradable cell delivery construct of the invention into said animal, wherein said cells secrete a therapeutic that is effective at ameliorating or treating said disease or condition.

The invention further provides a method of ameliorating or treating a disease or condition in an animal, including a human, comprising transplanting an effective amount of the therapeutic-containing microcapsules of the invention into said animal, wherein said therapeutic is effective at ameliorating or treating said disease or condition.

In these methods of treatment, the microcapsules or coated delivery constructs of the invention may be administered in an amount that would deliver sufficient therapeutic so as to be effective against the disease. For example, in the treatment of diabetes, a single mL of microcapsules would contain approximately 10,000-60,000 β islet equivalents and approximately 1-10 mL microcapsules would be implanted per kg body weight into a subject to secrete the required amount of insulin to control blood glucose levels.

A skilled practitioner would be able to test the secretion rate of the particular therapeutic from the microcapsules in vitro and, for any particular patient need, be able to calculate how many microcapsules would be required to treat that particular patient effectively.

The microcapsules of the invention may be formulated for allo- or xeno-transplantation depending on the source of the living cells and/or therapeutics.

The microcapsules of the invention may be transplanted within the tissues of the body or within fluid-filled spaces of the body, which ever is the most appropriate in terms of accessibility and efficacy. For example, if the living cells within the microcapsules are β islet cells, they may be transplanted in the peritoneal cavity. If the living cells with the microcapsules are choroid plexus cells and are for treating neurological disorders and any therapeutic agent secreted by the cells must be in contact with the cerebro spinal fluid surrounding the brain, such microcapsules may be implanted into or onto the brain.

Alternatively, the microcapsules may be formulated for oral or topical administration, particularly when they contain a therapeutic bioactive agent, such as an antibiotic.

The invention provides a use of an alginate containing between about 50 and about 95% mannuronic acid residues and a polycation in the manufacture of a microcapsule preparation for use in allo- or xeno-transplantation applications.

Such microcapsules may comprise living cells comprising naturally occurring or genetically or genetically engineered cells which may be in he form of single cells and/or cell clusters selected from the group comprising of β islet cells, hepatocytes, neuronal cells such as choroid plexus cells, pituitary cells, chromaffin cells, chondrocytes and any other cell type capable of secreting factors that would be useful in the treatment of a disease or condition.

Alternatively the microcapsules may comprise a therapeutic agent.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### EXAMPLES

The invention consists in the foregoing and also envisages constructions of which the following gives examples only. The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Intraperitoneal Stability of Alginate-Polyornithine Microcapsules in Rats

### An FTIR and SEM Analysis

### Materials and Methods

### Study Design

Monodisperse alginate-PLO microcapsules were fabricated from 5 different types of alginate and injected into the peritoneal cavity of Long-Evans rats. Prior to transplantation, the materials were characterized in vitro for the ratio of mannuronic acid to guluronic acid (M:G Ratio), endotoxin and protein levels, viscosity, and molecular weight. After 14, 30, 60, and 90 days, capsules were retrieved from each animal. The geometry of the retrieved capsules was assessed and the capsules were analyzed for chemical integrity by Fourier-Transform Infrared spectroscopy (FTIR) and surface morphology by scanning electron microscopy (SEM).

### Encapsulation Materials Source and Purification

Lyophilized alginate was purchased from 5 sources either in raw or purified form. 2 sources were provided in purified form by the manufacturer (see below) and the other 3 were received raw and subsequently purified using a solvent extraction method (33). Briefly, a 1% (W/V) solution was dissolved in a 1.0 mM sodium EGTA solution and filtered through successively more restrictive membranes (5.0, 1.5, 0.8, 0.45, and 0.22 μm filters). pH was lowered gradually to 1.5 and the precipitated alginate was washed three times in 0.01N HCl+20 mM NaCl. Using chloroform and butanol, proteins were extracted 3 times during a 30 minute exposure with vigorous shaking. After returning to neutral pH, the organic extraction was repeated and the alginate was precipitated in ethanol, filtered, washed with diethyl ether, and lyophilized for at least 72 hours. Prior to microcapsule formation, all alginates were dissolved in 1.5% (W/V) solutions in calcium and magnesium-free phosphate buffered saline (PBS) (Gibco, USA) and passed through a 0.22 μm filter for sterility. The alginates were designated as vendor-purified medium G (VPMG), vendor-purified low G (VPLG), purified Keltone LVCR (pKel), purified Fluka (pFlu), and purified Manucol (pMan) based on the approximated G-fraction specified by the suppliers. Keltone and Manucol alginates were obtained from ISP Corporation (USA) while Fluka was ordered from Sigma-Aldrich.

Polyomithine hydrobromide (MW=5-15 KDa, Sigma-Aldrich, USA) was dissolved in calcium and magnesium-free PBS and sterile filtered immediately prior to capsule fabrication. All other encapsulation reagents, including calcium chloride, sodium citrate, and sodium chloride, were purchased from Sigma-Aldrich and were made as sterile solutions on the day of encapsulation.

### Encapsulation Materials Alginate Characterization

Alginates were analyzed using a variety of techniques to distinguish important chemical properties including nuclear magnetic resonance spectroscopy (NMR), FTIR, viscometry, and gel permeation chromatography (GPC). The relative levels of protein and endotoxin were also determined for each alginate solution.

### NMR Spectroscopy

NMR spectroscopy was used to determine the ratio of mannuronic acid to guluronic acid residues in the carbohydrate copolymer. Samples were partially hydrolyzed to reduce viscosity and allow for the appropriate resolution on NMR as described by Grasdalen et al (34). Briefly, 1.0% (w/v) alginate was brought to pH 3.0 and maintained under reflux at 100° C. for 30 minutes. A Buchi Rotavapor (Switzerland) was used to remove the majority of the water while the remainder was lyophilized to complete dryness. Samples were then dissolved (20 mg/mL) in deuterium oxide and were analyzed on a Bruker NMR (300 MHz at 90° C.). The elevated temperature effectively shifted the water peak downfield to reveal the peaks of interest for integration. Bruker XWIN-NMR was used to measure the area under these peaks (δ≈5.7 ppm for G1, 5.3 ppm for M1 and GM5, and 4.9 ppm for GG5). The ratio of the area under G1 divided by the area under M1/GM5+GG5 was calculated to give the G-fraction. Samples were run in triplicate throughout the process.

### FTIR Spectroscopy

A Perkin-Elmer Series 1600 FTIR was used attached to a horizontal attenuated total reflectance (HATR) accessory for all measurements. Alginate powder or lyophilized microcapsules were placed onto the ZnSe crystal until it was fully covered and 100 psi pressure was applied to the sample. Scans from 4000-650 cm⁻¹ were acquired (N=32) and ATR correction was applied to the resulting spectra. Quantitative assessment was made by measuring the area under peaks of interest with the Perkin-Elmer Spectrum 5 software (35).

To characterize and quantify the effect of increasing PLO on the resultant spectra, PLO:Alginate was precipitated together with the concentration of PLO at 80%, 60%, 40%, 35%, 30%, 25%, 21%, 17%, 10%, and 5% (W/W). To achieve a homogeneous sample, a PLO solution in dH₂O was placed on top of frozen alginate aliquots. Using probe sonication, the PLO was gradually reacted and precipitated with the thawing alginate until the entire mixture was thawed and opaque. Sonication was carried out until a homogeneously opaque solution was obtained. Next, samples were flash-frozen in liquid nitrogen and immediately lyophilized. These dry samples were run in triplicate with spectra averaged over N=32 scans.

### Viscometry

Viscosity was measured using a Brookfield Cone/Plate Viscometer. The gap between the cup and the spindle was set for 0.013 mm prior to each run to eliminate noise related to sample level. 1 mL of 1.0% (W/V) alginate sample was added to the sample cup and distributed in a thin layer across the bottom of the cup in a manner that excluded air bubbles. The spindle was inserted to assess rotational resistance at a variety of speeds ranging from 1 to 20 rpm. Torque at the different shear speeds ranged from 25-95% within the optimum working range of the viscometer. Dynamic viscosity was calculated by the change in resistance verses the speed of the probe. All measurements were carried out at room temperature (25° C.).

### GPC

Alginate samples were dissolved at a concentration of 0.17% (W/V) and 50 μL was injected into a Waters (USA) Ultrahydrogel Linear Column affixed to a Perkin-Elmer GPC apparatus with an Isocratic 250 pump, 101 Oven, LC30 RI detector, and 900 series interface. Calibration standards used were poly(ethylene oxide) at molecular weights of 932, 571, 177, and 70 KDa dissolved in PBS buffer also at 0.17% (W/V). Using Nelson Turbochrom software, M_{w}, Mₙ, and M_{z} were calculated. The polydispersity index, or degree of polymorphism in molecular weight species, was calculated as M_{w}/Mₙ.

### Alginate Protein Content

Total protein in alginate samples was measured by the Micro BCA Protein Assay (Pierce, USA). Following spike and recover experiments with roughly 100% accuracy and dilution linearity of about 95%, 1 mL 1.7% (W/V) alginate samples were diluted 2, 5, 10, and 20-fold and were incubated with the working reagent for 2 hours at 37° C. for development. The developed reagent was detected on a 96-well plate with a UV-Vis spectrophotometer at 562 nM and quantified against a linear standard curve with bovine serum albumin.

### Endotoxin Content

The Limulus Amebocyte Lysate (LAL) CL-1000 Chromogenic LAL Endpoint Assay (Cambrex, USA) was used to quantify the total endotoxin content of the alginates under study. 1.7% (W/V) samples were incubated at a 10-fold dilution in dH₂O for 18 hours at 50° C. for endotoxin extraction and reacted over a defined time course against standard concentrations (36). Endpoint product was analyzed on a Beckman-Coulter DTX-880 UV-VIS Spectraphotometer. The assay had a detection range of 1-50 EU/mL.

### Alginate Microencapsulation

A 60-cc syringe was used to collect 30 mL of 1.7% (W/V) sterilized alginate solution that was affixed to the Inotech IE-50R electrostatic encapsulation machine (Switzerland). A syringe pump operating at roughly 8 mL/min was used to feed the solution through the nozzle vibrating at approximately 900 Hz. Due to the differences in the inherent viscosity of the various alginate solutions, these parameters were varied slightly as needed to maintain optimal machine operation. The fluid stream passed through an electrostatic ring with an applied current of approximately 1.5 kV and into a bath of 300 mL 100 mM CaCl₂ with 50 mM NaCl stirring without vortex. After crosslinking for 5 minutes, capsules were removed and immediately reacted with 100 mL 0.05% (W/V) PLO for 10 minutes followed by 2 washes in 3-(N-morpholino) propanesulfonic acid (MOPS) buffer. An outer alginate coat was then applied by stirring the capsules in 0.05% alginate for 5 minutes and the coated capsules were washed twice again in MOPS buffer. Capsules were prepared fresh and brought to 37° C. in 1 mL aliquots in sterile PBS prior to implantation. Aliquots were retained for pre-implantation analyses.

### Capsule Characterization Microscopy and Image Analysis

Capsule geometry was characterized by phase contrast light microscopy in conjunction with Scion Image (USA) morphometry. Capsules suspended in PBS were placed into 24-well plates, ensuring that only one layer of capsules remained on the bottom of the well. Using a 5× lens with phase contrast, images with large fields and clearly defined capsule outlines were obtained. At the same resolution, an image of a hemocytometer was acquired for calibration against a known distance. In Scion Image, the calibration image was used to set the appropriate scale and capsule diameters were measured approximating the maximum diameter and minimum diameter in case of spherical deviation. For simplification to a 2-D parameter, % cross-sectional uniformity was measured as the area based on the smaller radius divided by the area based on the larger radius X 100. At least 100 capsules were measured in each group at every timepoint.

### Aninmal Use

Male Long-Evans rats weighing between 250-350 g were housed in pairs and kept in a controlled environment with a 12:12 hour light-dark cycle. All animal use and handling was conducted under strict standards that either met or exceeded NIH guidelines. In addition, all procedures were approved in advance by the Brown University IACUC governing body. There were 5 animals in each material group (N=5) within each timepoint (N=4) for a total of 100 animals in the study.

Rats were anesthetized transiently with 3% isoflurane gas and 1 mL capsule volume suspended in 1 mL calcium- and magnesium-free PBS (for 2 mL total volume) was administered through a 16-gauge needle into the peritoneum at the midline. Animals were recovered and returned to cages at the termination of the procedure. Time 0 (pre-implantation) material and image analysis cohorts were also passed through a 16-gauge needle.

At 14, 30, 60, and 90 days after implantation, animals were sacrificed with CO₂ overdose and the capsules were retrieved under microscopy using a transfer pipette and PBS to collect free-floating capsules from all quadrants. Location, abundance, and gross appearance were documented. Next, pooled samples were characterized using image analysis, washed, and flash frozen for lyophilization.

### Post-Explant Capsule Characterization

Following a 72-hour period of lyophilization, capsules were analyzed using FTIR and SEM for surface chemistry and morphology. A similar procedure was used for FTIR as previously described for raw materials, except lyophilized beads were visually inspected for integrity in order to limit the analysis to the external surface of the capsule and not the bulk, and to confirm that adherent cells were flaked off to minimize tissue interference. Roughly 20 capsules were placed onto the ATR crystal to complete coverage and spectra were acquired at 100 psi. Multiple spectra from different capsules were acquired to confirm homogeneity of the sample population and combined.

Samples for SEM were placed on aluminum mounts lined with adhesive-coated carbon discs and were sputter-coated with a gold-palladium target under vacuum in an argon atmosphere. Coated specimens were examined on a Hitachi 2700 at an accelerating voltage of 5 to 8 kV. Digital capture images were used throughout.

### Results

### Pre-Encapsulation Characterization

Alginate materials were characterized prior to encapsulation based on their respective M:G ratios, protein and endotoxin levels, viscosity, and molecular weight. The results are shown in Table 1 below:

TABLE 1
Pre-encapsulation alginate characterization. Manufacturer specifications are
included for comparison.

| | | | | Total | | | | | |
|---|---|---|---|---|---|---|---|---|---|
| | | | | Protein | Endotoxin | | Molecular Weight | | |
| Alginate | | | M:G | Content | Level | Viscosity | (KDa) | | |
| Type | Source | Specifications | Ratio | (μg/mL) | (EU/mL) | (Cp) | M_{w} | M_{w}/Mₙ | M_{z} |
| VPMG | N/A | Low Viscosity | 56:44 | 31 | <1 | 25 | 317 | 3.9 | 840 |
| VPLG | N/A | Low Viscosity | 72:28 | 40 | <1 | 22 | 383 | 3.5 | 979 |
| pKel | Keltone | Medium G | 73:27 | 41 | 7.9 | 37 | 398 | 4.1 | 1163 |
| | LVCR | Low Viscosity | | | | | | | |
| pFlu | Fluka | High G | 13:87 | 34 | 39.5 | 45 | 534 | 6.3 | 1510 |
| pMan | Manucol | Low G | 49:51 | 86 | 40.5 | 88 | 609 | 14.5 | |
| | LKX | Medium | | | | | | | |
| | | Viscosity | | | | | | | |

In general, the rough specifications supplied by the manufacturer were similar to the results obtained from NMR with the exception that the pMan alginate was higher in guluronic acid content than expected. Viscosity, an indicator of molecular weight, was similar between groups as measured by dynamic viscosity. The 2 commercially-purified alginates had the lowest viscosity at 1.0% and 25° C. (VPMG: 25 Cp; VPLG: 22 Cp) while the alginates purified in house had increasing higher viscosities, respectively. Protein was also relatively consistent between all groups except for the pMan, which, at 86 μg/mL, had at least twice the amount of the other materials. Endotoxin levels trended similarly with the highest levels observed in the pFlu and pMan groups.

The peaks integrated from NMR spectra acquired at 90° C. are shown in FIG. 1. This spectrum, obtained from one of the samples at 90° C., shows the three peaks described earlier, where peak A represents the proton resonance at G1, peak B for M1 and GM5, and peak C for GG5. The peak due to protons present in the solvent, HDO, is shown at 4.7 ppm. It should be noted that the entire spectra at 90° C., aside from the HDO peak, was shifted downfield 0.8 ppm compared to room temperature conditions to further elucidate the alginate peaks.

Molecular weights were assessed by GPC and are shown in Table 1 above. In general, the weight-average molecular weight, M_{w}, correlated well with viscosity as predicted by the Sakurade-Houwink equation [η=KM^{α}]. pMan had the highest molecular weight at 609 KDa while the other groups ranged between 317-534 KDa. As expected with naturally synthesized biopolymers, the polydispersity index, or M_{w}/Mₙ of these samples showed some variability of sample polymorphism. The VPMG, VPLG, and pKel groups showed the narrowest distributions here while pFlu and pMan had the highest polydispersity.

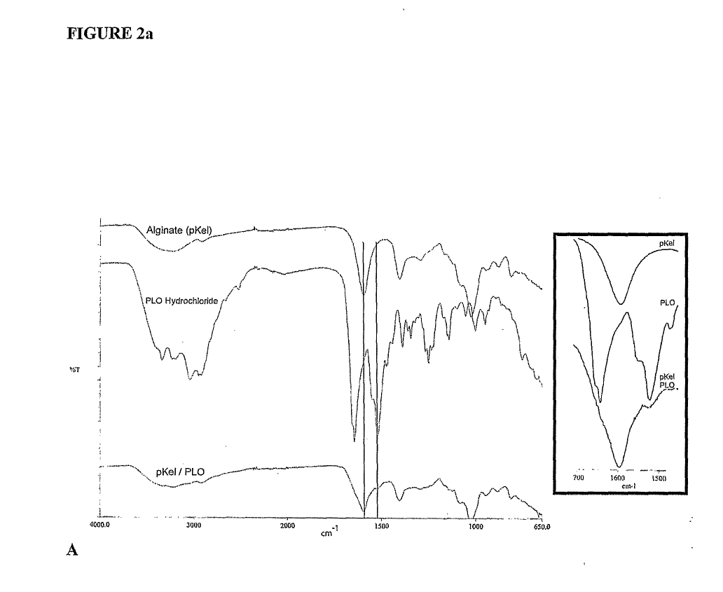

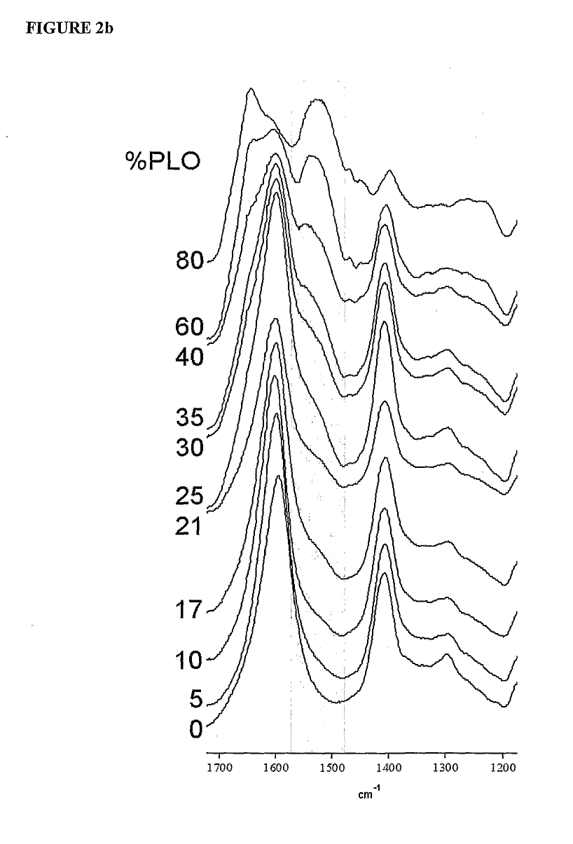

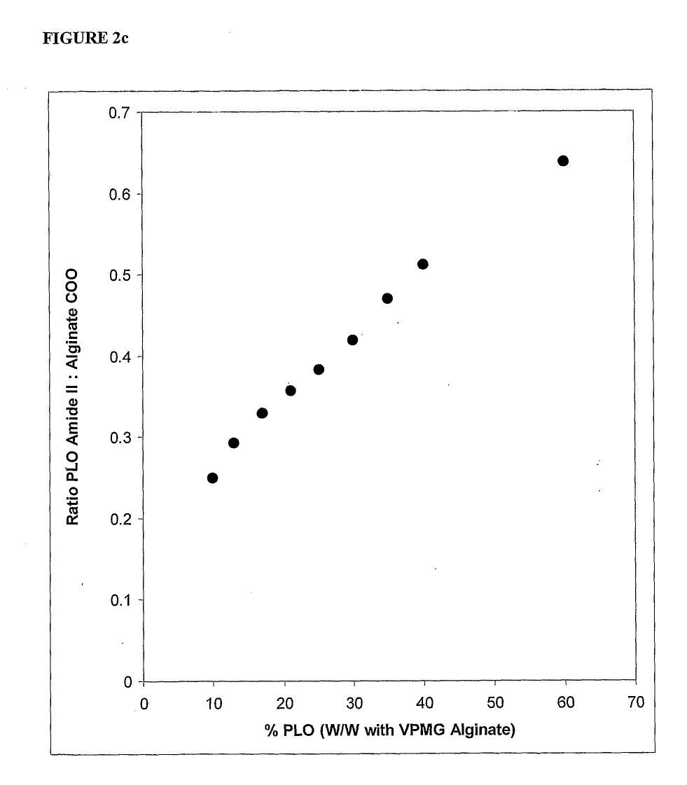

ATR-FTIR was used to characterize functional groups. In addition to using absorption values from previously reported findings using FTIR to study alginate and polycation capsules (35), we confirmed the range of reported findings on homogeneous lyophilized PLO:Alginate precipitates as well as the raw starting materials. This was carried out to characterize the relationship between the two components on the outer surface of the capsule for proper assessment of surface changes over time. The raw spectra, shown in FIG. 2a, reveal a number of peaks in both samples. Table 2, below, lists some of the relevant peaks detected in these samples and associated functional groups in alginate and alginate-PLO. FIG. 2b shows the differences in critical areas of interest, particularly in the carbonyl region associated with the Amide II bond of PLO (1550 cm⁻¹) and the carboxylic acid portion of the uronic acids (1590 cm⁻¹). Differences exist in the —NH and —CH₂ absorptions of Alginate/PLO compared to alginate alone but at a lower magnitude.

TABLE 2
FTIR peaks in Alginate and Alginate-PLO samples. Corresponding
functional groups are shown in the far right column.

| Peak Location | | | |
|---|---|---|---|
| (cm⁻¹) | Alginate | Alginate-PLO | Functional Group |
| 3400 | | | —OH |
| 3062 | | | —NH |
| 2920 | | | —CH₂ |
| 1590/1640 | 1590 | 1640 (PLO), 1590 | —COO⁻ |
| | | (Alginate) | |
| 1550 | | | -Amide II |
| 1403 | | | —COO⁻ |
| 1167 | | | —COC, —OH |
| 1122 | | | —CO, —CC |
| 1085 | | | —CO, —CCO, —CC |
| 1027 | | | —CO, —CC, —COH |

The effect of increasing PLO on the sample surface is shown in FIGS. 2c and 2d. The spectra shown in FIG. 2c show decreasing amplitude of the peak related to the PLO Amide II absorption at 1550 cm⁻¹ as the PLO in the sample is reduced. Quantitatively, this relationship can be expressed by the ratio of the area under the curve of the Amide II absorption to the Alginate COO⁻ absorption. As PLO is increased in the sample, this ratio increases linearly as shown in FIG. 2d. These samples lack calcium while the explanted capsules retain it, which can affect spectral shift (35) and the exact position of the absorption peak. Regardless, this observation signifies that small changes in relative composition can be detected with this method.

### Microcapsule Characterization

Capsules freshly collected following encapsulation were analyzed based on geometry and morphology prior to lyophilization. Diameter, cross-sectional uniformity, and wall thickness were measured using image analysis (Table 3 below). Microcapsule formulations were similar in size and spanned a range of roughly 170 μm in diameter. Similarly, the range of wall thickness was narrow ranging from 18.0 to 19.7 μm.

TABLE 3
Geometric evaluation of microcapsules prior to implantation.

| | Diameter (μm) | | |
|---|---|---|---|
| Alginate | (±standard | Cross-Sectional | Wall Thickness (μm) |
| Material | deviation) | Uniformity (%) | (±standard deviation) |
| VPMG | 596 ± 0.7 | 100 | 18.4 ± 0.7 |
| VPLG | 694 ± 0.5 | 100 | 19.6 ± 1.5 |
| pKel | 766 ± 2.2 | 100 | 19.7 ± 1.6 |
| pFlu | 670 ± 0.6 | 100 | 18.4 ± 1.4 |
| pMan | 660 ± 10.6 | 100 | 18.0 ± 2.7 |

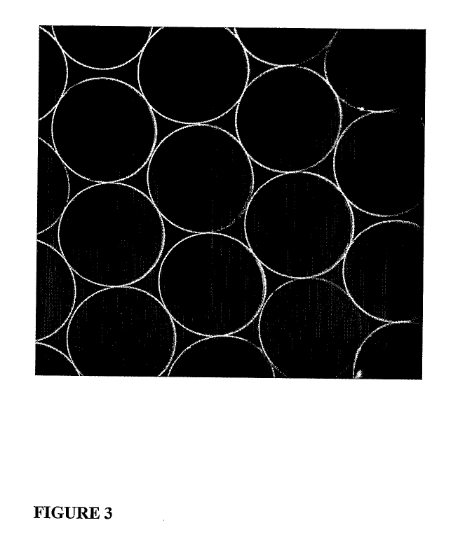

Pre-implant capsule morphology was characterized by well-rounded, smooth surfaces with homogeneous size distributions within each sample population. Cross-sectional thickness was constant throughout the perimeter of the capsule wall and no gross defects were noted in any sample group. The most monodisperse capsule population at the experimental onset was the VPLG group with only 0.07% variation while the pMan group varied the most but only at 1.6%. No obvious morphologic differences, aside from diameter, were observed between groups. A representative sample of a starting dose capsules (VPMG) is shown in FIG. 3 in a phase-contrast micrograph. Here, the symmetry and monodisperse nature of the capsule preparation is apparent. This is highly representative of all groups at the onset of the experiment.

### Gross Observations and Geometry of Explanted Microcapsules

Capsules implanted into the peritoneum were found localized to the omenta, porta hepatis, intestinal mesentery, and pelvis in all groups at all time points. Occasionally, aggregates were found in close proximity to the liver or the posterior abdominal wall. In the latter case, capsule aggregates existed as 2-D cakes and 3-D clusters. Only capsules retrieved in a free-floating manner were used for FTIR and SEM characterization, which accounted for the bulk of samples.

At day 14, all animals contained free-floating individual capsules localized to the areas described above. At day 14, a marked decrease in the clarity and round shape of the capsules was observed in the pFlu and pMan groups, which continued to become more apparent at each successive time point.

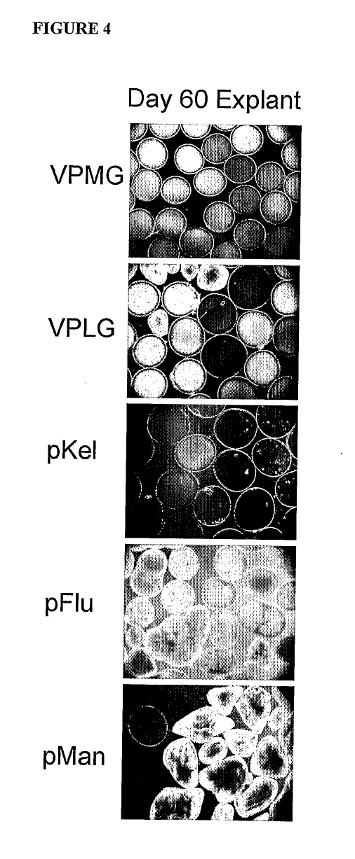

After 90 days of implantation, the pMan group was difficult to retrieve as most capsules were found in amorphous aggregates of 1-3 with no defined shape or 3-D architecture. pFlu became more amorphous over the same time frame but to a lesser extent than pMan. This apparent change in morphology was observed at day 60 in both the pKel and VPLG groups, where a portion of the population showed deformation in addition to mild opacification of the interior. In contrast, the VPMG group maintained morphology for the duration of the experiment and even on day 90 showed no surface gross irregularity indicative of stability loss. In this group, there was some internal opacification present starting at day 60. For comparison, representative micrographs immediately following explanation on day 60 are shown in FIG. 4.

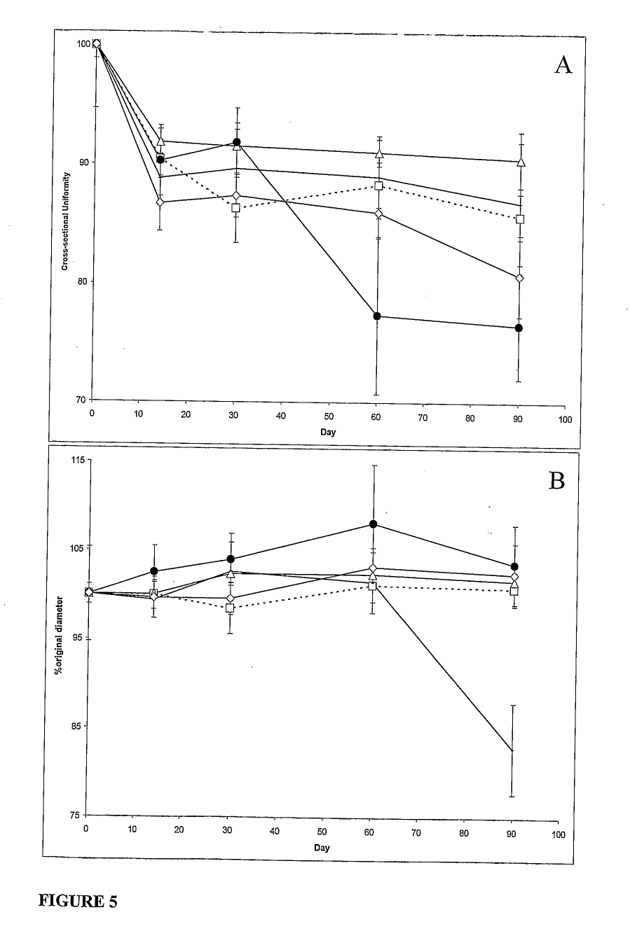

Capsules were measured to characterize the change in diameter as well as cross-sectional uniformity, or concentricity, over time. The data are plotted in FIG. 5. The cross-sectional uniformity, shown in FIG. 5A, was initially 100% in all groups, indicating that the starting product was completely concentric. Over time, this value drops notably in each group although the VPMG group maintained over 90% for the duration. The change in cross-sectional uniformity can be attributed to the deformation that occurred as the material degraded, losing stability and becoming more susceptible to physical stress. The magnitude of this change was greatest in the pMan, pFlu, and VPLG groups. The change in diameter over time is shown in FIG. 5B. All of the groups showed a small increase in diameter over time, with the pMan group exhibiting the most significant increase, to 108% by 60 days. The change in diameter can initially be attributed to swelling of the hydrogel matrix but, as cross-sectional uniformity decreases and some groups undergo deformation, this too affects the overall diameter. This is likely the cause of the large increase in the pMan group. Finally, the pKel group, which exhibited a reduction in diameter between 60 and 90 days, supports a degradation mechanism leading to capsule deflation.

### FTIR Analysis of Explanted Microcapsule Surface

ATR-FTIR was carried out on the surface of capsules from each group at the time of explant following lyophilization. As confirmed visually and shown on electron microscopy, cells adhering to the surface were detached in the lyophilization process and thus did not interfere with the surface. The spectra generated from raw materials in addition to information reported elsewhere (35) was used to compare the capsules over time. Specifically, as mentioned previously and highlighted in Table 2, above, the peaks at 1590 cm⁻¹/1640 cm⁻¹ and 1550 cm⁻¹ (alginate COO⁻ and polyornithine COO⁻/Amide II respectively) were used to differentiate the surface chemistry of the outermost layer. The other peaks related to polyornthine exposure, for example the —NH peak at 3062 cm⁻¹ and the —CH₂ peak at 2920 cm⁻¹, were of lower intensity and thus were more difficult to obtain repeatable quantitative results from integration.

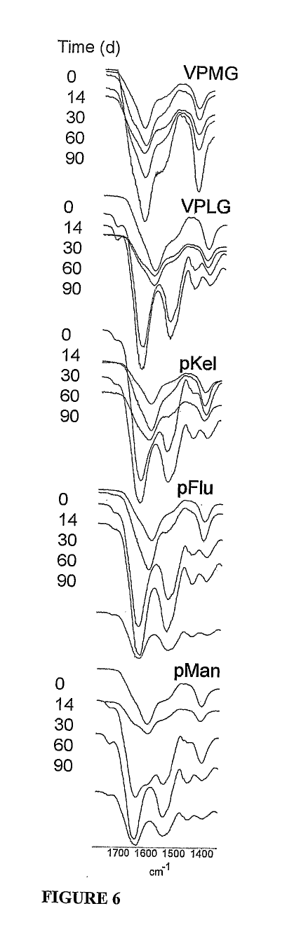

The relevant peaks are shown in comparison in FIG. 6. The peaks are displayed over time from time 0 (top) to 90 days (bottom). In all groups except for the VPMG group, the small shoulder due to the Amide II component of polyornithine on the surface became a distinct second peak and the PLO peak at 1640 cm¹ emerged, indicating surface erosion of alginate and prominence of PLO on the surface. In the case of pMan and pFlu, this emergence is clear by day 30 and reaches its maximum amplitude by day 60. pKel and VPLG provide additional stability as the Amide II peak does not fully emerge until day 60. Importantly, the VPMG group maintains a consistent surface chemistry as evidenced by the Amide II shoulder at day 90. Its amplitude does increase slowly over time but a fully discrete peak is not observed.

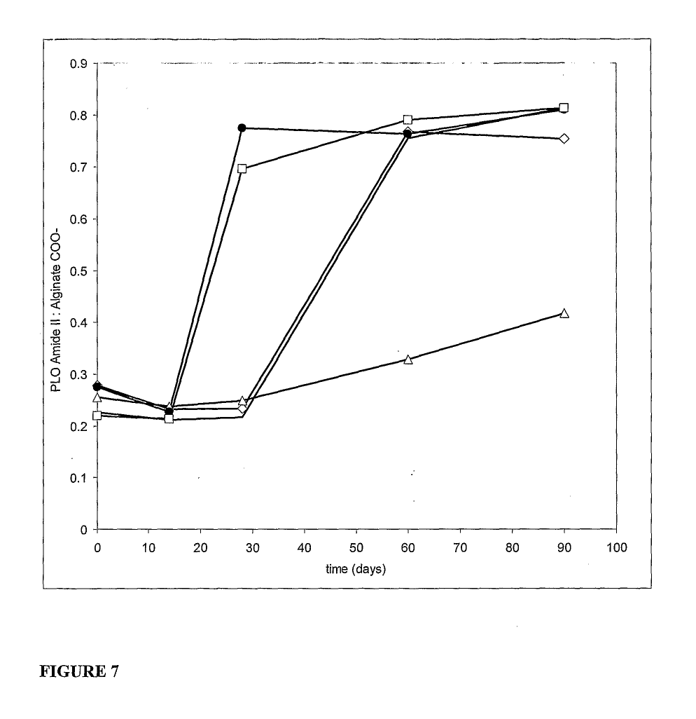

To quantitatively characterize the changes in these chemical absorptions, the area under the alginate —COO⁻ and polyornithine—Amide II peaks were integrated and compared over time. The ratio of the area under the alginate peak to the area under the polyornithine peak was calculated and is displayed in FIG. 7. A relative stability index can be assigned to this value correlative of the amount of alginate degradation on the surface with the assumption that the amount of alginate on the surface compared to the amount of PLO on the surface is related to the ratio of these two peaks, to a point of total disappearance and a emergence of the PLO carboxylic peak at 1640 cm⁻¹. The utility of using this index as a measure of how much of each wall is present on the surface is exemplified by the fact that bulk PLO:Alginate samples demonstrated linearity between composition and this ratio, and these capsules are composed of distinct walls of alginate and PLO.

As shown in FIG. 7, the ratio of these peaks starts at a similar value for all groups (0.25±0.03) and demonstrates uniformity at time 0. The change in the amplitude of the peaks over 90 days (FIG. 6) is directly reflected in the index calculated here. There are 3 groups of materials, those that degrade rapidly by 30 days (pMan, pFlu), those that maintain stability to 60 days (VPLG, pKel), and those that are stable for the duration of the experiment (VPMG). All of the groups except for VPMG experience a modest decrease initially and then increase to roughly 0.7 to 0.8. The stability index of VPMG exhibits a slow, continual increase throughout the time period to 0.4 at 90 days. This gradual increase in the relative proportion of polyornithine functional groups to alginate functional groups is linear and may be indicative of a surface erosion mechanism.

### Explanted Microcapsule Morphological Analysis: SEM Analysis of Explanted Microcapsule Morphology

Lyophilized cohorts were coated and the surface was scanned using SEM. Bulk capsule analysis was initially carried out at a magnification of 100-200× followed by microanalysis of the surface at 1000-2000×. High magnification images were acquired at 5000-15000× as required and as permitted by material. In cases of degraded materials, it was often not possible to achieve such high magnification due to damage of the material by the electron beam. In some cases, debris from the lyophilization process was unavoidable and was included in certain images.

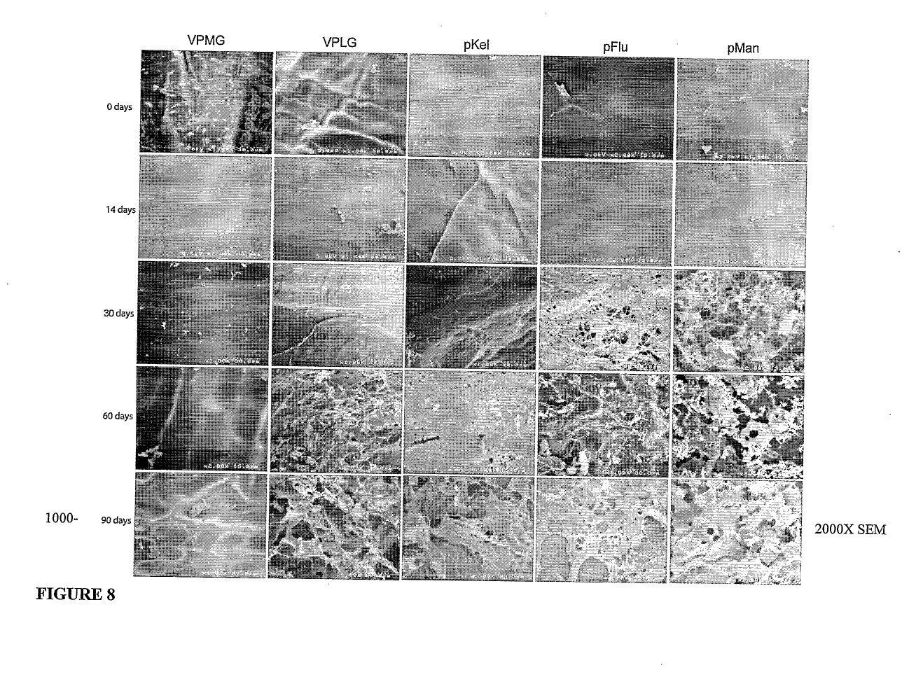

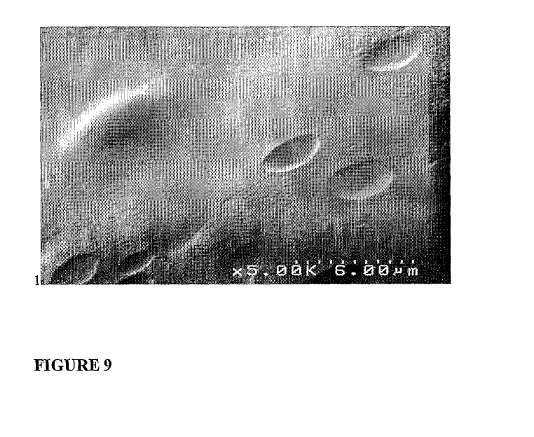

Intermediate magnification images (1-2K X) were used for the bulk of the comparative analysis and are presented in FIG. 8. In general, these findings support the gross morphologic observations from phase-contrast light microscopy and the FTIR analysis of the surface stability. The magnifications used further allowed visualization of micro-pitting on the surface and slight changes in morphology. All groups had extremely smooth surfaces over the first 14 days followed by the appearance of surface defects at various time points. pMan and pFlu both showed extreme degeneration of the surface by 30 days with continued erosion until 90 days. The small holes in the surface at day 30 became increasingly larger and discrete alginate and PLO layers were separated. The initiation of this erosion probably occurred between day 14 and 30 but was not captured morphologically. pKel and VPLG showed a similar progression of surface erosion, however the 30 day timepoint revealed the onset of degradation in the form of surface pits. These pits, shown in high magnification in FIG. 9, progressed to small holes that continued to increase in size through day 90. VPMG maintained a completely smooth surface through the duration of the experiment although the level of apparent wrinkling of the surface increased at day 60 and further at day 90. This is likely artifact of the lyophilization process but may be related to the physical integrity of the capsule over time. The other materials were so highly degraded at these time points that it is probable that such gross deformation would be masked.

### Example 2

### Characteristics of the Polycation PLO (poly-L-ornithine)

The polyanionic core of calcium alginate requires a polycationic coating to contribute to the strength and the semipermeable characteristics of the biocompatible capsule. The polycation exists as a mixed population of molecular species of varying lengths and hence of varying molecular weights. Studies were conducted to determine the preferred molecular weight species of PLO. Biocapsules were made as described in Example 1 using different batches of PLO to obtain capsules wherein the encapsulated cells or beads were centrally placed and the capsule wall not compromised.

High MW Species: As summarized in the Table 4 below, biocapsules were optimally intact when the composition of the PLO did not contain high molecular weight species above 42 KDa. PLO of average MW of 42 KDa and 56 KDa produced unacceptable capsules which adhered to each other forming clumps.

TABLE 4
Optimal Capsules using PLO of low Molecular Weight

| PLO Average Mw | Position of encapsulated cells | Integrity of Capsule |
|---|---|---|
| 23 KDa | Cells in central position and not protruding | Pockets noted in 2% of |
| Fill: SLO1674 | into the capsule wall, only 6% of capsules had | capsules. No clumping of |
| | cells in the periphery but none protruding onto | capsules |
| | capsule wall | |
| 42 KDa | Cells central position and not protruding into | Clumping of capsules |
| | capsule wall | |
| 56.4 KDa | Cells central position and not protruding into | Clumping of capsules |
| | capsule wall | |

Extremely Low MW Species: A poorly performing batch of PLO of expected MW of 23 KDa was subjected to dialysis using a dialysis cassette with a membrane molecular weight cut off of 10 KDa (Pierce, Slide-A-Lyzer Dialysis Cassette, Gamma Irradiated, 10K MZCO, 12-30 ml, Rockford, Ill. 61105, USA). Superior capsules were obtained with the PLO batch which had been dialysed to remove polypeptides of less than 10 KDa. See Table 5.

TABLE 5
Optimal Capsules obtained using PLO without extremely low molecular weight species

| PLO Average Mw | Position of encapsulated cells | Integrity of Capsule |
|---|---|---|
| 23 KDa | 50% of capsules with cells in the periphery | Pockets noted within 40-50% |
| Lot 82K | and encroaching on the capsule wall and in | of capsules |
| Fill * 3K * HK | 5% of capsules cell clusters protrude into the | |
| with low Mw species | capsule wall | |
| 23 KDa | Most cells were central and not protruding | Pockets noted in 10-15% |
| Lot 82K | into the capsule wall. Only 10% of capsules | of capsules |
| Fill * 3K * HK | had cells in the periphery and in less than 2% | |
| Post dialysis with | were cells encroaching on the capsule wall | |
| 10 KDa cut off | | |

Molecular Weight Polydispersity: An analysis of the polydispersity of PLO batches showed that the polycation is supplied as a mixture of polypeptides with a range of molecular weights (Table 6). Based on the quality of biocapsules made with different batches of PLO, the molecular weight distribution profile of a PLO batch should exclude molecular species at the extremes of the molecular size range. It is concluded that the optimal PLO composition had a polydispersity ratio (defined as the ratio of the average Mw to the median Mw) of less than 1.5, and preferably less than 1.1.

TABLE 6
Polydispersity (MW/MN) of PLO

| | MALLS | | | | | | | | | | | |
|---|---|---|---|---|---|---|---|---|---|---|---|---|
| Sigma | Analysis | | % Mass in given MW Range in KDa | | | | | | | | | |
| Reference | MW | MW/MN | <1 | 1-5 | 5-10 | 10-15 | 15-20 | 20-25 | 25-30 | 30-40 | 40-100 | >100 |
| 2533 | 11.6 | 1.15 | 0 | 4.8 | 36.9 | 43.3 | 10.7 | 1.9 | 0.7 | 0.8 | 1.7 | 0.1 |
| 3655 | 13.4 | 1.54 | 0 | 0 | 15.9 | 45.7 | 21.3 | 5.4 | 3.1 | 4.2 | 4.4 | 0.1 |
| 3655 | 35.7 | 1.55 | 0 | 0 | 0.2 | 2.4 | 7.6 | 11 | 12.2 | 16.7 | 38.2 | 11.7 |
| 5666 | 1.79 | 1.6 | 39.8 | 57.8 | 1.5 | 0.3 | 0.2 | 0.2 | 0.1 | 0.1 | 0.2 | 0.1 |

### DISCUSSION

Purified alginate with the highest levels of mannuronic acid residues (VPMG) and a polycationic agent having a polydispersity index of less than 1.5 produced microcapsules which are superior to other prior art microcapsules, as well as to the other purified alginates tested, in terms of capsule geometry and their durability and functionality in vivo.

### INDUSTRIAL APPLICATION

The compositions of the present invention are useful in the formation of immunoisolatory microcapsules for use in delivering living cells capable of secreting therapeutics, or to deliver therapeutics per se, for the treatment of diseases or disorders.

It is not the intention to limit the scope of the invention to the abovementioned examples only. As would be appreciated by a skilled person in the art, many variations are possible without departing from the scope of the invention as defined in the accompanying claims.

### REFERENCES

1. Murphy, G. P.; Mundy, R. L. Ewald, R. A. The Physiologic Properties Of Glyco-Alginate, A New Japanese Plasma-Volume Expander. Surgery. 56: 1099-108; 1964.
2. Kierstan, M. Bucke, C. The immobilization of microbial cells, subcellular organelles, and enzymes in calcium alginate gels. Biotechnol Bioeng. 19: 387-97; 1977.
3. Fan, M. Y.; Lum, Z. P.; Fu, X. W.; Levesque, L.; Tai, I. T. Sun, A. M. Reversal of diabetes in BB rats by transplantation of encapsulated pancreatic islets. Diabetes. 39: 519-22; 1990.
4. Fritschy, W. M.; Wolters, G. H. van Schilfgaarde, R. Effect of alginate-polylysine-alginate microencapsulation on in vitro insulin release from rat pancreatic islets. Diabetes. 40: 37-43; 1991.
5. Hobbs, H. A.; Kendall, W. F., Jr.; Darrabie, M. Opara, E. C. Prevention of morphological changes in alginate microcapsules for islet xenotransplantation. J Investing Med. 49: 572-5; 2001.
6. Korbutt, G. S.; Mallett, A. G.; Ao, Z.; Flashner, M. Rajotte, R. V. Improved survival of microencapsulated islets during in vitro culture and enhanced metabolic function following transplantation. Diabetologia. 47: 1810-8; 2004.
7. Leung, Y. F.; O'Shea, G. M.; Goosen, M. F. Sun, A. M. Microencapsulation of crystalline insulin or islets of Langerhans: an insulin diffusion study. Artif Organs. 7: 208-12; 1983.
8. Schrezenmeir, J.; Kirchgessner, J.; Gero, L.; Kunz, L. A.; Beyer, J. Mueller-Klieser, W. Effect of microencapsulation on oxygen distribution in islets organs. Transplantation. 57: 1308-14; 1994.
9. Trivedi, N.; Keegan, M.; Steil, G. M.; Hollister-Lock, J.; Hasenkamp, W. M.; Colton, C. K.; Bonner-Weir, S. Weir, G. C. Islets in alginate macrobeads reverse diabetes despite minimal acute insulin secretory responses. Transplantation. 71: 203-11; 2001.
10. Wang, T.; Lacik, I.; Brissova, M.; Anilkumar, A. V.; Prokop, A.; Hunkeler, D.; Green, R.; Shahrokhi, K. Powers, A. C. An encapsulation system for the immunoisolation of pancreatic islets. Nat. Biotechnol. 15: 358-62; 1997.
11. Joseph, J. M.; Goddard, M. B.; Mills, J.; Padrun, V.; Zurn, A.; Zielinski, B.; Favre, J.; Gardaz, J. P.; Mosimann, F.; Sagen, J. et al. Transplantation of encapsulated bovine chromaffin cells in the sheep subarachnoid space: a preclinical study for the treatment of cancer pain. Cell Transplant. 3: 355-64; 1994.
12. Garcia-Martin, C.; Chuah, M. K.; Van Damme, A.; Robinson, K. E.; Vanzieleghem, B.; SaintRemy, J. M.; Gallardo, D.; Ofosu, F. A.; Vandendriessche, T. Hortelano, G. Therapeutic levels of human factor VIII in mice implanted with encapsulated cells: potential for gene therapy of haemophilia A. J Gene Med. 4: 215-23; 2002.
13. Hortelano, G.; Al-Hendy, A.; Ofosu, F. A. Chang, P. L. Delivery of human factor IX in mice by encapsulated recombinant myoblasts: a novel approach towards allogeneic gene therapy of hemophilia B. Blood. 87: 5095-103; 1996.
14. Aebischer, P.; Tresco, P. A.; Sagen, J. Winn, S. R. Transplantation of microencapsulated bovine chromaffin cells reduces lesion-induced rotational asymmetry in rats. Brain Res. 560: 43-9; 1991.
15. Borlongan, C. V.; Skinner, S. J.; Geaney, M.; Vasconcellos, A. V.; Elliott, R. B. Emerich, D. F. Neuroprotection by encapsulated choroid plexus in a rodent model of Huntington's disease. Neuroreport. 15: 2521-5; 2004.
16. Borlongan, C. V.; Skinner, S. J.; Geaney, M.; Vasconcellos, A. V.; Elliott, R. B. Emerich, D. F. Intracerebral transplantation of porcine choroid plexus provides structural and functional neuroprotection in a rodent model of stroke. Stroke. 35: 2206-10; 2004.
17. Borlongan, C. V.; Skinner, S. J.; Geaney, M.; Vasconcellos, A. V.; Elliott, R. B. Emerich, D. F. CNS grafts of rat choroid plexus protect against cerebral ischemia in adult rats. Neuroreport. 15: 1543-7; 2004.
18. Maysinger, D.; Piccardo, P.; Filipovic-Grcic, J. Cuello, A. C. Microencapsulation of genetically engineered fibroblasts secreting nerve growth factor. Neurochem Int. 23: 123-9; 1993.
19. Maysinger, D.; Piccardo, P.; Liberini, P.; Jalsenjak, I. Cuello, C. Encapsulated genetically engineered fibroblasts: release of nerve growth factor and effects in vivo on recovery of cholinergic markers after devascularizing cortical lesions. Neurochem Int. 24: 495-503; 1994.
20. Ross, C. J. Chang, P. L. Development of small alginate microcapsules for recombinant gene product delivery to the rodent brain. J Biomater Sci Polym Ed. 13: 953-62; 2002.
21. Tresco, P. A.; Winn, S. R. Aebischer, P. Polymer encapsulated neurotransmitter secreting cells. Potential treatment for Parlinson's disease. Asaio J. 38: 17-23; 1992.
22. Visted, T.; Bjerkvig, R. Enger, P. O. Cell encapsulation technology as a therapeutic strategy for CNS malignancies. Neuro-oncol. 3: 201-10; 2001.
23. Winn, S. R.; Tresco, P. A.; Zielinski, B.; Greene, L. A.; Jaeger, C. B. Aebischer, P. Behavioral recovery following intrastriatal implantation of microencapsulated PC12 cells. Exp Neurol. 113: 322-9; 1991.
24. Xue, Y.; Gao, J.; Xi, Z.; Wang, Z.; Li, X.; Cui, X.; Luo, Y.; Li, C.; Wang, L.; Zhou, D.; Sun, R. Sun, A. M. Microencapsulated bovine chromaffin cell xenografts into hemiparkinsonian rats: a drug-induced rotational behavior and histological changes analysis. Artif Organs. 25: 131-5; 2001.
25. Boontheekul, T.; Kong, H. J. Mooney, D. J. Controlling alginate gel degradation utilizing partial oxidation and bimodal molecular weight distribution. Biomaterials. 26: 2455-65; 2005.
26. Bouhadir, K. H.; Lee, K. Y.; Alsberg, E.; Damm, K. L.; Anderson, K. W. Mooney, D. J. Degradation of partially oxidized alginate and its potential application for tissue engineering. Biotechnol Prog. 17: 945-50; 2001.
27. Kendall, W.; Darrabie, M.; Freeman, B.; Hobbs, H.; Collins, B. Opara, E. Effect of bead swelling on the durability of polylysine alginate microcapsules. Curr Surg. 57: 636-637; 2000.
28. Kendall, W. F., Jr.; Darrabie, M. D.; El-Shewy, H. M. Opara, E. C. Effect of alginate composition and purity on alginate microspheres. J Microencapsul. 21: 821-8; 2004.
29. Kong, H. J.; Kaigler, D.; Kim, K. Mooney, D. J. Controlling rigidity and degradation of alginate hydrogels via molecular weight distribution. Biomacromolecules. 5: 1720-7; 2004.
30. Lee, K. Y.; Bouhadir, K. H. Mooney, D. J. Controlled degradation of hydrogels using multifunctional cross-linking molecules. Biomaterials. 25: 2461-6; 2004.
31. De Vos, P.; De Haan, B. Van Schilfgaarde, R. Effect of the alginate composition on the biocompatibility of alginate-polylysine microcapsules. Biomaterials. 18: 273-8; 1997.
32. De Vos, P.; De Haan, B. J.; Wolters, G. H.; Strubbe, J. H. Van Schilfgaarde, R. Improved biocompatibility but limited graft survival after purification of alginate for microencapsulation of pancreatic islets. Diabetologia. 40: 262-70; 1997.
33. Bunger, C. M.; Gerlach, C.; Freier, T.; Schmitz, K. P.; Pilz, M.; Werner, C.; Jonas, L.; Schareck, W.; Hopt, U. T. de Vos, P. Biocompatibility and surface structure of chemically modified immunoisolating alginate-PLL capsules. J Biomed Mater Res A. 67: 1219-27; 2003.
34. Grasdalen, H.; Larsen, B. Smidsrod, O. A P.M.R. Study of the Composition and Sequence of Uronate Residues in Alginates. Carbohydrate Research. 68: 23-31; 1979.
35. van Hoogmoed, C. G.; Busscher, H. J. de Vos, P. Fourier transform infrared spectroscopy studies of alginate-PLL capsules with varying compositions. J Biomed Mater Res A. 67: 172-8; 2003.
36. Nakagawa, Y.; Murai, T.; Hasegawa, C.; Hirata, M.; Tsuchiya, T.; Yagami, T. Haishima, Y. Endotoxin Contamination in Would Dressings Made of Natural Biomaterials. J Biomed Mater Res. 66B: 347-355; 2003.

1-82. (canceled)

83. A composition comprising alginate and a polycation, wherein said polycation has an average molecular weight of 10 to 40 kDa and a polydispersity index of less than about 1.5 and wherein the polycation is not poly-L-lysine, and wherein the alginate has a mannuronic acid concentration selected from the group consisting of (i) between from about 50% to about 95% mannuronic acid residues, (ii) between from about 50% to about 90% mannuronic acid residues; (iii) between from about 50% to about 70% mannuronic acid residues, and (iv) between about 80% and about 90%.

84. The composition as claimed in claim 83, comprising 87% of a high mannuronic acid alginate.

85. The composition of claim 83, where the polycation is selected from the group consisting of chitosan glutamate, chitosan glycol, modified dextran, lysozyme, poly-L-ornithine, salmine sulfate, protamine sulfate, polyacrylimide, polyacrylimide-co-methacryloxyethyltrimethylammonium bromide, polyallylamine, polyamide, polyamine, polybrene, Polybutylacrylate-co-Methacryloxyethyl Trimethylammonium Bromide (80/20), Poly-3-chloro-2-hydroxypropylmethacryloxyethyl dimethylammonium Chloride, Polydiallyldimethylammonium, Polydiallyldimethylammonium Chloride, Polydiallyldimethylammonium Chloride-co-Acrylamide, Polydiallyldimethylammonium Chloride-co-N-Isopropyl Acrylamide, Polydimethylamine-co-epichlorohydrin, Polydimethylaminoethylacrylate-co-Acrylamide, Polydimethylaminoethylmethacrylate, Polydimethylaminoethyl Methacrylate, Polyethyleneimine, Polyethyleneimine-Epichlorohydrin Modified, Polyethyleneimine, Poly-2-hydroxy-3-methacryloxypropyl Trimethylammonium Chloride, Poly-2-hydroxy-3-methacryloxyethyl, Trimethylammonium Chloride, Polyhdroxyproplymethacryloxy Ethyldi methyl Ammonium Chloride, Polyimadazoline (Quaternary), Poly-2-methacryloxyethyltrimethylammonium Bromide, Polyniethacryloxyethyltrimethylammonium Bromide/Chloride, Polymethyldiethylaminoethylmethacrylate-co-Acrylamide, Poly-1-methyl-2-vinylpyridinium Bromide, Poly-1-methyl-4-vinylpyridinium Bromide, Polymethylene-co-Guanidine Hydrochloride, Polyvinylamine, Poly-N-vinylpyrrolidone-co-Dimethylaminoelhyl-Methacrylate, and Poly-4-vinylbenzyltrimethylammonium Chloride, and Poly-4-vinylbenzyltrimethylammonium Chloride.

86. The composition of claim 83, where in the polycation is poly-L-ornithine having an average molecular weight selected from (i) between about 10-40 KDa, (ii) is between about 15 and 30 KDa, and (iii) between 20 and 25 KDa and contains less than 20% of a molecular weight species of 10 KDa or less.

87. The composition of claim 83, wherein the ratio of mannuronic acid alginate to polycation is from about 5:1 to about 10:1.

88. The composition of claim 83, further comprising less than about 1% calcium chloride and/or sodium chloride.

89. A biocompatible microcapsule comprising a core layer of a high mannuronic acid alginate cross-linked with a cationic cross-linking agent, an intermediate layer of polycations forming a semi-permeable membrane, and an outer layer of a high mannuronic acid alginate, wherein the high mannuronic acid alginate in the core and outer layers is the same or different and contains between from about 50% to about 95% mannuronic acid residues, wherein the polycation layer is not comprised of poly-L-lysine.

90. The biocompatible microcapsule of claim 89, wherein the high mannuronic acid alginate has an average molecule weight of greater than about 400 KDa and the polycationic agent has an average molecular weight of between 10 and 40 KDa.

91. The biocompatible microcapsule of claim 90, wherein the high mannuronic acid alginate has an average molecular weight of greater than about 600 KDa and the polycationic agent has an average molecular weight of between 15 and 30 KDa.

92. The biocompatible microcapsule of claim 89, wherein the cross-linking agent is selected from salts of the group consisting of Ag⁺, Al³⁺, Ba²⁺, Ca²⁺, Cd²⁺, Cu²⁺, Fe²⁺, Fe³⁺, H⁺, K⁺, Li⁺, Mg²⁺, Mn²⁺, Na⁺, NH⁴⁺, Ni²⁺, Pb²⁺, Sn²⁺ and Zn²⁺.

93. The biocompatible microcapsule as claimed in claim 92, wherein the cross-linking agent is calcium chloride.

94. The biocompatible microcapsule of claim 89, wherein the intermediate layer is between about 10 and about 80 microns in thickness.

95. The biocompatible microcapsule of claim 89, wherein the core layer has been depolymerised by a chelation agent to form a hollow core.

96. The biocompatible microcapsule of claim 95, wherein the chelation agent is selected from sodium citrate and EDTA.

97. The biocompatible microcapsule of claim 89, wherein the ratio of the core layer to the intermediate layer is about 7:1 to about 8:1 by weight.

98. The biocompatible microcapsule of claim 89, wherein the ratio of the outer layer to the intermediate layer is about 1.2:1 to about 1.4:1 by weight.

99. The biocompatible microcapsule of claim 89, comprising living cells within the core layer.

100. The biocompatible microcapsule of claim 99, wherein the cells are present as single cells and/or cell clusters selected from the group consisting of β islet cells, hepatocytes, neuronal cells and any other cell type capable of secreting factors useful in the treatment of a disease or condition.

101. The biocompatible microcapsule of claim 100, wherein the neuronal cells are selected from the group comprising choroid plexus cells, pituitary cells, chromafin cells and chondrocytes.

102. The biocompatible microcapsule of claim 89 further comprising a therapeutic agent.

103. The biocompatible microcapsule of claim 89, having a diameter of between 50 and 2000 microns.

104. A method of preparing a biocompatible microcapsule comprising the steps:
a) dissolving a high mannuronic acid containing alginate in isotonic saline to a concentration of between about 1.0% to 2.0% w/v;
b) spraying the dissolved alginate solution of step a) through an air- or frequency-based droplet generator into a stirring solution of an excess of a cross-linking agent to form gelled capsules;
c) coating the gelled capsules of step b) with a polycation having an average molecular weight of 10-40 kDa and a polydispersity index of less than 1.5, wherein the polycation is not poly-L-lysine;
d) dissolving a high mannuronic acid alginate in isotonic saline to a concentration of about 0.01 to about 1.7% w/v and applying as a final coating to the capsule of step c); and
e) collecting the microcapsules;
wherein the high mannuronic acid-containing alginate of steps a) and d) is the same or different and contains between about 50% to about 95% mannuronic acid residues.

105. The method of claim 104, wherein the high mannuronic acid alginate solution of step (a) further comprises a small molecule, protein or a DNA molecule or a therapeutic agent.

106. The method of claim 104, wherein step b) comprises stirring in about 15 mM to about 120 mM calcium chloride for between about 5 to about 30 minutes.

107. The method of claim 106, wherein step b) comprises stirring in about 110 mM calcium chloride for between about 5 to about 10 minutes.

108. The method of claim 104, wherein step c) comprises coating the capsules with poly-L-ornithine at a concentration of between about 0.02% to about 0.10% (w/v) for between about 1 to about 45 minutes.

109. The method of claim 108, wherein the poly-L-ornithine has an average molecular weight of between about 10 and 40 KDa or between 15 and 30 KDa.

110. The method of claim 109, wherein the poly-L-ornithine has an average molecular weight of between 20 and 25 KDa and contains less than 20% of a molecular weight species of 10 KDa or less.

111. The method of claim 108, wherein step c) comprises coating the capsules with a poly-L-ornithine solution at a concentration of about 0.05% (w/v) for about 10 minutes.

112. The method of claim 104, wherein in step d) the final high mannuronic acid alginate coating solution is applied at a concentration of between 0.02% and about 1.0% w/v for between about 5 and about 30 minutes.

113. The method of claim 112, wherein step d) comprises applying a final high mannuronic acid alginate coating solution at a concentration of about 0.05% w/v for between 5 and about 10 minutes.

114. The method of claim 104, wherein the high mannuronic acid alginate solution of step a) and step d) is the same or different and comprises from about 50% to about 70% mannuronic acid residues.

115. A method of preparing microencapsulated cells comprising the steps:
a) incubating living cells in a solution of high mannuronic acid containing alginate dissolved in isotonic saline to a concentration of between about 1.0% and 2.0% w/v;
b) spraying the cell-containing alginate solution of step c) through an air- or frequency-based droplet generator into a stirring solution of an excess of a cross-linking agent to form gelled cell-containing capsules;
c) coating the gelled cell-containing capsules of step b) with a polycation having an average molecular weight of 10-40 kDa and a polydispersity index of less than 1.5;
d) dissolving a high mannuronic acid containing alginate in isotonic saline to a concentration of about 0.01 to about 1.7% w/v and applying as a final coating to the cell-containing capsules of step c); and
e) collecting the cell-containing microcapsules;
wherein the high mannuronic acid containing alginate of steps a) and d) is the same or different contains from about 50% to about 95% mannuronic acid residues; and wherein the polycation is not poly-L-lysine.

116. The method of claim 115, wherein step b) comprises stirring in about 15 mM to about 120 mM calcium chloride for between about 5 to about 30 minutes.

117. The method of claim 116, wherein step b) comprises stirring in about 110 mM calcium chloride for between about 5 to about 10 minutes.

118. The method of claim 115, wherein step c) comprises coating the capsules with poly-L-ornithine at a concentration of between about 0.02% to about 0.10% (w/v) for between about 1 to about 45 minutes.

119. The method of claim 118, wherein step c) comprises coating the capsules with poly-L-ornithine at a concentration of about 0.05% (w/v) for about 10 minutes.

120. The method of claim 117, wherein the poly-L-ornithine has an average molecular weight of between 10 and 40 KDa or between 15 and 30 KDa.

121. The method of claim 120, wherein the poly-L-ornithine has an average molecular weight of between 20 and 25 KDa and contains less than 20% of a molecular weight species of 10 KDa or less.

122. The method of claim 115, wherein in step d) the final high mannuronic acid alginate coating solution is applied at a concentration of between 0.02% and about 1.0% w/v for between about 5 and about 30 minutes.

123. The method of claim 122, wherein step d) comprises applying a final high mannuronic acid alginate coating solution at a concentration of about 0.05% w/v for between 5 and about 10 minutes.

124. The method of claim 115, wherein the high mannuronic acid alginate solution of step a) and step d) is the same or different and contains from about 50% to about 70% mannuronic acid residues.

125. A method for coating non-degradable cell delivery construct comprising the steps:
a) immersing the non-degradable cell delivery construct in a solution of high mannuronic acid containing alginate dissolved in isotonic saline to a concentration of between 1.0% to 2.0% w/v;
b) incubating the construct of step a) in a solution containing an excess of a cross-linking agent to form a gelled coating;
c) further coating the gelled construct of step b) with a polycation having an average molecular weight of 10-40 kDa and a polydispersity index of less than 1.5;
d) dissolving a high mannuronic acid containing alginate in isotonic saline to a concentration of from about 0.01 to about 1.7% w/v and applying as a final coat to produce an immunoisolatory membrane coated non-degradable cell delivery construct; and
e) isolating the immunoisolatory membrane coated non-degradable cell delivery construct;
wherein the high mannuronic acid containing alginate of steps a) and d) is the same or different and contains between about 50% to about 95% mannuronic acid residues; and wherein the polycation is not poly-L-lysine.

126. The method of claim 125, wherein step b) comprises stirring in about 15 mM to about 120 mM calcium chloride for between about 5 to about 30 minutes.

127. The method of claim 126, wherein step b) comprises stirring in about 110 mM calcium chloride for between about 5 to about 10 minutes.

128. The method of claim 125, wherein step c) comprises coating the capsules with poly-L-ornithine at a concentration of between about 0.02% to about 0.10% (w/v) for between about 1 to about 45 minutes.

129. The method of claim 128, wherein step c) comprises coating the capsules with poly-L-ornithine at a concentration of about 0.05% (w/v) for about 10 minutes.

130. The method of claim 128, wherein the poly-L-ornithine has an average molecular weight of
(i) between 10 and 40 KDa,
(ii) between 15 and 30 KDa, and
(iii) between 20 and 25 KDa and contains less than 20% of a molecular weight species of 10 KDa or less.

131. The method of claim 125, wherein in step d) the final high mannuronic acid alginate coating solution is applied at a concentration of between 0.02% and about 1.0% w/v for between about 5 and about 30 minutes.

132. The method of claim 131, wherein step d) comprises applying a final high mannuronic acid alginate coating solution at a concentration of about 0.05% w/v for between 5 and about 10 minutes.

133. The method of claim 125, wherein the high mannuronic acid alginate solution of step a) and step d) is the same or different and contains from about 50% to about 70% mannuronic acid residues.

134. The method of claim 125, wherein step c) comprises coating the capsules with poly-L-ornithine at a concentration of about 0.05% (w/v) for about 10 minutes.

135. A biocompatible microcapsule prepared by the method of claim 101.

136. The cell-containing microcapsule prepared by the method of claim 115.

137. An immuno-isolatory membrane coated non-degradable cell delivery construct prepared by the method of claim 125.

138. A method of ameliorating or treating a disease or condition in a subject comprising transplanting an effective amount of the therapeutic-containing microcapsule of claim 99 in the said subject, when said therapeutic is effective at ameliorating or treating said disease or condition.

139. A method of ameliorating or treating a disease or condition in a subject comprising transplanting an effective amount of a cell-containing microcapsule of claim 99 into said subject, when said cells secrete a therapeutic that is effective at ameliorating or treating said disease or condition.

140. The method of claim 139, wherein said living cells comprise β islet cells and said disease or condition is diabetes.

141. The method of claim 139, wherein said living cells comprise hepatocytes and said disease or condition is a disease or disorder of the liver.

142. The method of claim 139, wherein said living cells comprise neuronal cells selected from the group consisting of choroid plexus cells, pituitary cells, chromafin cells, chondrocytes and any other neuronal cell capable of secreting neuronal factors, and the disease or condition is a neurological disease or condition.
